(19) United States
(12) Patent Application Publication
(10) Pub. No.: US 2008/0293053 A1
(43) Pub. Date: Nov. 27, 2008

(54) shRNA Materials and Methods of Using Same for Inhibition of DKK-1

(75) Inventors: Evan T. Keller, Ann Arbor, MI (US); Christopher L. Hall, Ypsilanti, MI (US); Stuart A. Aaronson, New York, NY (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: THE REGENTS OF THE UNIVERSITY OF MICHIGAN, Ann Arbor, MI (US)

(21) Appl. No.: 11/966,665

(22) Filed: Dec. 28, 2007

Related U.S. Application Data
(60) Provisional application No. 60/877,499, filed on Dec. 28, 2006.

(51) Int. Cl. C12Q 1/68; C12N 5/06; C12N 15/00; C07H 21/04

(52) U.S. Cl. 435/6; 435/375; 435/320.1; 536/23.1; 435/325

(57) ABSTRACT

Methods and compositions for the treatment of soft tissue cancer are described. More specifically, the invention demonstrates that inhibiting or otherwise decreasing the activity of DKK-1 using shRNA or siRNA molecules will be effective at reducing the cancer phenotype of prostate cancer cells.

This invention was made with government support under Grant No. P01 CA093900 and R01CA071672. awarded by the National Cancer Institute. The government has certain rights in the invention.

### FIELD OF THE INVENTION

The present invention relates to methods and compositions for the treatment of prostate cancer. More particularly, the invention is directed to inhibiting cancer cell growth, and/or proliferation, and/or metastases and/or promoting prostate cancer cell apoptosis comprising administering shRNA and siRNA molecules directed against DKK-1.

### BACKGROUND OF THE INVENTION

Prostate cancer is the second leading cause of cancer-related deaths in men resulting in over 30,000 deaths annually. More than 80% of all men who die of prostate cancer have metastatic disease within the bone. Growth of prostate cancer within the bone promotes localized bone turnover that results in primarily osteoblastic (increased bone density) lesions with underlying osteopenic (low bone density) lesions. Although mechanisms contributing to the osteopenic component of prostate cancer-mediated bone lesions have been elucidated, the mechanisms responsible for the osteoblastic component of prostate cancer bone lesions remain unknown. Several proteins including endothelins and bone morphogenetic proteins have been hypothesized to play roles in osteoblastic lesions; however, there are no published data showing that they mediate prostate cancer-induced osteoblastic lesions in vivo.

Wnt proteins are soluble glycoproteins that bind to receptor complexes composed of Lrp5/6 and Frizzled proteins. Wnt-mediated signaling promotes postnatal bone accrual. Additionally, analysis of both chick and mouse limb development has shown that expression of Wnt proteins is essential for skeletal outgrowth. The activity of the Wnt family is antagonized by several secreted factors including dickkopf (DKK), Wnt inducible factor-1, secreted frizzled-related proteins, and cerberus. DKK-1 controls Wnt signaling by binding the LRP coreceptor and sterically blocking Wnt binding to the receptor complex. DKK-1 modulation of Wnt signals is also required to achieve normal limb development in vertebrates. Recently, the expression of DKK-1 was found in osteolytic foci of multiple myeloma suggesting that cancer-mediated modulation of Wnt activity influences bone remodeling.

In one study, investigators tested whether the balance between Wnts and a Wnt antagonist influences the osteoblastic phenotype of prostate cancer-induced bone lesions. In that study, it was shown that Wnt2 was increased in prostate cancer metastases versus primary lesions and both Wnt 5a and Wnt 6 were increased in prostate cancer versus normal prostate. Wnt 1, Wnt 2b, Wnt 4, Wnt 5b, Wnt 7a, Wnt 8b, Wnt 9b, Wnt 10a, Wnt 10b, and Wnt 11 mRNA levels were not different among tumor versus normal prostate or metastases versus primary tumors. Importantly, that study also showed that DKK-1 expression was decreased in prostate cancer versus normal prostate tissue. In the osteolytic PC-3 cells, DKK-1 mRNA and protein was most highly expressed in the parental PC-3 cell line and decreased with increasing malignancy. These data were found to be consistent with the relative decrease of DKK-1 expression levels observed in the clinical specimens. This led to the conclusion that as prostate cancer progresses, DKK-1 expression level decreases and suggest that as the cell line becomes osteoblastic that DKK-1 expression is decreased. Even when shRNA was employed to decrease DIK-1 expression in PC3 prostate cancer cells, there was no difference in cell proliferation between shRNA control versus DKK-1 shRNA clones. As such, no anti-tumor effects were seen with shRNAs targeted to DKK-1.

In additional studies, it has been shown that DKK-1 is a tumor suppressor. Its expression was shown to decrease 56% of human colorectal cancer. Expression of DDK-1 in colorectal cancer cells also suppressed sub-Q tumor growth. DKK-3, a DKK-like molecule also has tumor suppressor activity, and it was found to be down-regulated in human hepatoma samples, and its expression hepatocellular carcinoma cells suppressed colony formation in vitro and reduced tumor growth in vivo.

U.S. 2006/0003953 provides examples of DKK-1 related antisense molecules and methods of using the same for modulating DKK-1 expression in order to promote bone growth. Disruption of the interaction between DKK-1 and wnts also is contemplated for use in modulating bone mass and osteoporosis (U.S. 2005/0070699; U.S. 2004/0244069; and U.S. 2004/0221326). DKK-3 and DKK-3 related proteins and nucleic acids are described in U.S. 2003/0068312, which further states that in hyperproliferative disorders can be treated by administering DKK-related proteins. DKK-1 is thought in that document to be useful for the treatment of placental disorders. U.S. Pat. No. 6,344,541 discusses “DKR polypeptides” as being human orthologs of DKK-1 and suggests the use of DKK-1 polypeptides as having utility as anticancer agents. Use of DKK-1 proteins also were suggested for inducing neurogenesis, enhancing proliferation, self-renewal, survival and/or dompaminergic induction, differentiation and the like (WO 2006/061717). The involvement of various has been postulated for monitoring beta cell dysfunction in diabetes (WO 03/032810; WO 02/066509). Antibodies and peptides to DKK-1 also have been discussed and described in WO 2006/015373. Other documents describing the preparation of DKK-1 proteins include WO 2005/112981; WO 2005/049797; WO 2005/049640; WO 2004/053063; and WO 00/52047

To date, however, there has been no suggestion or indication that inhibition of DKK-1 expression using shRNA and/or siRNA molecules will be useful in producing an anti-tumor effect on prostate cancer cells. Indeed, given that it has been shown that DKK-1 is a tumor suppressor and that its expression in hepatocellular carcinoma cell suppressed colony formation in vitro and reduced tumor growth in vivo, those skilled in the art have predicted that inhibition of DKK-1 would have an effect of increasing the tumorigenicity, cancer growth, and/or cancer cell proliferation and/or decreasing prostate cancer cell apoptosis rather than being beneficial in the treatment of prostate cancer.

### SUMMARY OF THE INVENTION

The present invention is directed to methods and compositions for the treatment of prostate cancer, by inhibiting the expression of DKK-1 in the prostate cells by contacting the prostate cancer cells with a composition that comprises an shRNA or an siRNA molecule directed against said DKK-1.

Thus, the present invention provides a method of reducing tumor burden from prostate cancer cells comprising the step of contacting the prostate cancer cells with a composition comprising an shRNA directed against DKK-1.

Also provided by the invention is a method of reducing the metastasis of prostate cancer cells comprising contacting the prostate cancer cells with a composition that comprises an shRNA directed against DKK-1.

Further provided by the invention is a method of treating prostate cancer in a patient comprising administering to the patient a composition comprising a vector encoding a shRNA directed against DKK-1, wherein the vector is taken up by prostate cancer cells in said patient and said shRNA is expressed in an amount sufficient to block the expression of DKK-1 in said prostate cancer cells.

In an embodiment of the invention, prostate cancer cells are contacted with an anti-cancer agent.

In an aspect of the invention, the shRNA is directed against a sequence selected from one or more of the sequences of SEQ ID NO:2, SEQ ID NO:3, SEQ ID NO:4, SEQ ID NO:5, SEQ ID NO:10, SEQ ID NO:11, SEQ ID NO:12, SEQ ID NO:13 and SEQ ID NO:14.

In another aspect of the invention, the shRNA consists of a sequence selected from the group consisting of SEQ ID NO:6, SEQ ID NO:7, SEQ ID NO:8, SEQ ID NO:9, SEQ ID NO: 15, SEQ ID NO:16, SEQ ID NO:17, SEQ ID NO:18 and SEQ ID NO:19.

In another aspect of the invention, a composition comprising a vector encoding an shRNA sequence directed against a DKK-1-encoding polynucleotide sequence selected from a group consisting of one or more of the sequences of SEQ ID NO:2, SEQ ID NO:3, SEQ ID NO:4, SEQ ID NO:5, SEQ ID NO: 10, SEQ ID NO:11, SEQ ID NO:12, SEQ ID NO:13 and SEQ ID NO:14.

In an embodiment, the shRNA of the invention is selected from the group consisting of SEQ ID NO:6, SEQ ID NO:7, SEQ ID NO:8, SEQ ID NO:9, SEQ ID NO: 15, SEQ ID NO:16, SEQ ID NO:17, SEQ ID NO:18 and SEQ ID NO:19.

In another embodiment, the invention provides a mammalian host cell stably transfected with a vector that drives transcription of a polynucleotide encoding an shRNA sequence directed against a DKK-1-encoding polynucleotide sequence selected from a group consisting of one or more of the sequences of SEQ ID NO:2, SEQ ID NO:3, SEQ ID NO:4, SEQ ID NO:5, SEQ ID NO: 10, SEQ ID NO:11, SEQ ID NO:12, SEQ ID NO:13 and SEQ ID NO:14.

In yet another embodiment, the invention provides a mammalian host cell stably transfected with a vector that drives transcription of a polynucleotide encoding an shRNA selected from the group consisting of SEQ ID NO:6, SEQ ID NO:7, SEQ ID NO:8, SEQ ID NO:9, SEQ ID NO: 15, SEQ ID NO:16, SEQ ID NO:17, SEQ ID NO:18 and SEQ ID NO:19.

The present invention further provides a method for diagnosing prostate cancer comprising the step of detecting DKK-1 activity levels in a patient, wherein elevated levels compared to levels in a normal individual are suggestive of prostate cancer, and wherein said normal individual is known not to suffer from prostate cancer.

Also provided by the present invention is a method for diagnosing prostate cancer comprising the step of detecting DKK-1 activity levels in a patient, wherein elevated levels compared to prior levels in said patient are suggestive of prostate cancer.

The present invention also provides a method for determining susceptibility to prostate cancer in a patient, comprising the step of determining DKK-1 activity levels in said patient, wherein elevated DKK-1 activity levels in said patient compared to DKK-1 activity levels in a normal individual indicate susceptibility to prostate cancer, wherein said normal individual is known not to suffer from prostate cancer.

Further provided by the present invention is a method for determining the progression of prostate cancer in a patient, comprising the step of determining DKK-1 activity levels in said patient, wherein increasing levels over time are indicative of prostate cancer progression.

Also provided by the present invention is a method for monitoring the effectiveness of prostate cancer treatment comprising the step of measuring DKK-1 activity over time, wherein a decrease in a rate of DKK-1 activity increase is indicative of effective treatment.

The present invention also provides a method for determining susceptibility to bone cancer in a patient, comprising the step of determining DKK-1 activity in said patient, wherein elevated DKK-1 activity in said patient compared to DKK-1 activity in a normal individual indicates susceptibility to bone cancer, and wherein said normal individual is known not to suffer from bone cancer.

Further provided by the present invention is a method for determining susceptibility to bone cancer in a patient, comprising the step of determining DKK-1 activity in said patient, wherein elevated DKK-1 activity in said patient compared to prior levels in said patient indicates susceptibility to bone cancer.

In an embodiment, DKK-1 mRNA levels are used to measure DKK-1 activity in a patient tissue sample to determine susceptibility to bone cancer, or monitor the effectiveness of prostate cancer treatment, or determine the susceptibility to prostate cancer, or determine the progression of prostate cancer, or to diagnose prostate cancer.

In another embodiment, DKK-1 protein levels are used to measure DKK-1 activity in a patient tissue sample to determine susceptibility to bone cancer, or monitor the effectiveness of prostate cancer treatment, or determine the susceptibility to prostate cancer, or determine the progression of prostate cancer, or to diagnose prostate cancer.

In still another embodiment, DKK-1 enzyme activity levels are measured in a patient tissue sample to determine susceptibility to bone cancer, or monitor the effectiveness of prostate cancer treatment, or determine the susceptibility to prostate cancer, or determine the progression of prostate cancer, or to diagnose prostate cancer.

In an embodiment, the patient tissue sample is selected from the group consisting of a bodily fluid, tissue or organ of said patient.

Further scope of the applicability of the present invention will become apparent from the detailed description provided below. However, it should be understood that the following detailed description and examples, while indicating preferred embodiments of the invention, are given by way of illustration only since various changes and modifications within the spirit and scope of the invention will become apparent to those skilled in the art from this detailed description.

### DETAILED DESCRIPTION OF THE PREFERRED EMBODIMENTS OF THE INVENTION

It is suggested herein that blocking DKK-1 activity will be a therapeutic target for the prevention of prostate cancer and/or its bone metastasis. In the present invention it is postulated that shRNA and/or siRNA molecules directed against DKK-1 can be used to achieve the relevant blocking of DKK-1. As used herein, a molecule that is “directed against DKK-1” is understood to mean a molecule that hybridizes to a polynucleotide encoding DKK-1, wherein such hybridization results in a decrease in DKK-1 activity. Alternatively, a molecule that is “directed against DKK-1” is understood to mean a molecule that hybridizes to a polynucleotide encoding DKK-1, wherein such hybridization results in an inhibition of DKK-1 translation. Any such molecule that blocks the expression or activity of the DKK-1 in prostate cells will be a therapeutic agent for use in the treatment of prostate cancer.

In the present invention, short hairpin RNA (shRNA) technology is used to reduce expression of DKK-1 in prostate cancer cells. Transfection of ˜21 nucleotide small interfering RNAs (siRNAs) can be used to transiently knock down the expression of specific genes in mammalian cells. In order to obtain long-term gene silencing, expression vectors that continually express siRNAs in stably transfected mammalian cells can be used (Brummelkamp et al., Science 296: 550-553, 2002; Lee et al., Nature Biotechnol. 20:500-505, 2002; Miyagishi, M, and Taira, K. Nature Biotechnol. 20:497-500, 2002; Paddison, et al., Genes & Dev. 16:948-958, 2002; Paul et al., Nature Biotechnol. 20:505-508, 2002; Sui, Proc. Natl. Acad. Sci. USA 99(6):5515-5520, et al., 2002; Yu et al., Proc. Natl. Acad. Sci. USA 99(9):6047-6052, 2002). Many of these plasmids have been engineered to express shRNAs lacking poly (A) tails. Transcription of shRNAs is initiated at a polymerase III (pol III) promoter and is believed to be terminated at position 2 of a 4-5-thymine transcription termination site. Upon expression, shRNAs are thought to fold into a stem-loop structure with 3′ UU-overhangs. Subsequently, the ends of these shRNAs are processed, converting the shRNAs into ˜21 nt siRNA-like molecules. The siRNA-like molecules can, in turn, bring about gene-specific silencing in the transfected mammalian cells.

Thus, shRNAs can be used for the treatment of disease states in which it is desirable to reduce or eliminate the expression of a particular gene. The length of the stem and loop of functional shRNAs can be varied. Stem lengths could range anywhere from 25 to 29 nucleotides and loop size could range between 4 to 23 nucleotides without affecting silencing activity. Moreover, presence of G-U mismatches between the two strands of the shRNA stem does not necessarily lead to a decrease in potency. Complementarity between the portion of the stem that binds to the target mRNA (antisense strand) and the mRNA, on the other hand, was shown to be critical. Single base mismatches between the antisense strand of the stem and the mRNA abolished silencing. In addition, the presence of 2 nucleotide 3′-overhangs is critical for siRNA activity. Presence of overhangs on s RNAs, however, did not seem to be important. Some of the functional shRNAs that were either chemically synthesized or in vitro transcribed, for example, did not have predicted 3′ overhangs.

With the above parameters in mind, the present invention contemplates that preparation of expression vectors and/or chemically synthesized shRNA molecules that can be delivered to the desired prostate cancer cell to effect the therapeutic effect of reducing the DKK-1 mRNA and/or reducing DKK-1 protein levels in the prostate cancer cells.

Thus, provided are nucleic acid and nucleic acid-like oligomers, which are targeted to a nucleic acid encoding DKK-1. These compounds are useful as modulators of the expression of a DKK-1. This invention also provides methods of modulating the expression of DKK-1 in cells, tissues or animals comprising contacting said cells, tissues or animals with one or more of the compounds or compositions of the present invention. For example, in one embodiment, the compounds or compositions of the present invention can be used to inhibit the expression of DKK-1 in cells, tissues or animals and particularly in the prostate tissue or cells of the animals.

Modulation of expression of the target DKK-1 nucleic acid can be achieved through alteration of any number of nucleic acid (DNA or RNA) functions. In the present case, the shRNA molecules are used to effect a decrease (inhibition or reduction) in DKK-1 expression. “Expression” includes all the functions by which the DKK-1 gene's coded information is converted into structures present and operating in a cell. These structures include the products of transcription and translation. “Modulation of expression” means the perturbation of such functions. The functions of DNA to be modulated can include replication and transcription. Replication and transcription, for example, can be from an endogenous cellular template, a vector, a plasmid construct or otherwise. The functions of RNA to be modulated can include translocation functions, which include, but are not limited to, translocation of the RNA to a site of protein translation, translocation of the RNA to sites within the cell which are distant from the site of RNA synthesis, and translation of protein from the RNA. RNA processing functions that can be modulated include, but are not limited to, splicing of the RNA to yield one or more RNA species, capping of the RNA, 3′ maturation of the RNA and catalytic activity or complex formation involving the RNA which may be engaged in or facilitated by the RNA. Modulation of expression can result in the increased level of one or more nucleic acid species or the decreased level of one or more nucleic acid species, either temporally or by net steady state level. It is a goal of the present invention to produce a therapeutically effective decrease in target DKK-1 RNA or protein levels in prostate cancer such that the proliferation, growth or metastasis of the cancer cell is reduced and/or the apoptosis of the cancer cell is increased. In another embodiment the shRNA molecules of the invention also may produce a decrease of one or more RNA DKK-1 splice products, or a change in the ratio of two or more splice products.

For therapeutic purposes, an animal, such as a human, that presents with a prostate cancer is treated by administering the shRNA and/or siRNA compounds in accordance with this invention. For example, the methods comprise the step of administering to said animal, a therapeutically effective amount of an antisense compound that inhibits expression of DKK-1 in order to promote apoptosis of the prostate cancer cell, and/or prevent the cancer cell from metastasizing, and/or prevent the cancer cell from growing and/or prevent the cancer cell from proliferating. Compounds of the invention can be used to modulate the expression of DKK-1 in any animal, and preferably in a human. In some embodiments, the methods comprise the step of administering to said animal an effective amount of an antisense compound that inhibits expression of DKK-1. Because reduction in DKK-1 mRNA levels can lead to alteration in DKK-1 protein products of expression as well, such resultant alterations can also be measured. shRNA compounds of the present invention that effectively produce an appreciable level of inhibition of the levels or function of DKK-1 RNA or protein products of expression will be considered active antisense compounds of the invention. In one embodiment, the compounds of the invention inhibit the expression of DKK-1 causing a reduction of DKK-1 RNA by at least 10%, by at least 20%, by at least 25%, by at least 30%, by at least 40%, by at least 50%, by at least 60%, by at least 70%, by at least 75%, by at least 80%, by at least 85%, by at least 90%, by at least 95%, by at least 98%, by at least 99%, or by 100% in the prostate cancer cells of the subject.

The reduction of the expression of DKK-1 can be measured in a bodily fluid, tissue or organ of the animal. Bodily fluids include, but are not limited to, blood (serum or plasma), lymphatic fluid, cerebrospinal fluid, semen, urine, synovial fluid and saliva and can be obtained by methods routine to those skilled in the art. Tissues or organs include, but are not limited to, blood (e.g., hematopoietic cells, such as human hematopoietic progenitor cells, human hematopoietic stem cells, CD34+ cells CD4+ cells), lymphocytes and other blood lineage cells, skin, bone marrow, spleen, thymus, lymph node, brain, spinal cord, heart, skeletal muscle, liver, pancreas, prostate, kidney, lung, oral mucosa, esophagus, stomach, ilium, small intestine, colon, bladder, cervix, ovary, testis, mammary gland, adrenal gland, and adipose (white and brown). Samples of tissues or organs can be routinely obtained by biopsy. In some alternative situations, samples of tissues or organs can be recovered from an animal after death.

The cells contained within said fluids, tissues or organs being analyzed can contain a nucleic acid molecule encoding DKK-1 protein and/or the encoded DKK-1 protein itself. For example, fluids, tissues or organs procured from an animal can be evaluated for expression levels of the target mRNA or protein. mRNA levels can be measured or evaluated by real-time PCR, Northern blot, in situ hybridization or DNA array analysis. Protein levels can be measured or evaluated by ELISA, immunoblotting, quantitative protein assays, protein activity assays (for example, caspase activity assays) immunohistochemistry or immunocytochemistry. Furthermore, the effects of treatment can be assessed by measuring biomarkers associated with the target gene expression in the aforementioned fluids, tissues or organs, collected from an animal contacted with one or more compounds of the invention, by routine clinical methods known in the art. These biomarkers include but are not limited to: glucose, cholesterol, lipoproteins, triglycerides, free fatty acids and other markers of glucose and lipid metabolism; liver transaminases, bilirubin, albumin, blood urea nitrogen, creatine and other markers of kidney and liver function; interleukins, tumor necrosis factors, intracellular adhesion molecules, C-reactive protein and other markers of inflammation; testosterone, estrogen and other hormones; tumor markers; vitamins, minerals and electrolytes.

In specific embodiments, the compositions of the invention are prepared as nucleic acid vectors and are contacted directly with prostate tissue to effect an immediate and local uptake of the shRNA composition at the site at which the action is desired.

The compositions of the invention also can be presented as combinations. For example, it is contemplated that in the treatment methods more than one shRNA molecule directed against DKK-1 is used. Thus the compositions can contain two or more oligomeric compounds. In another related embodiment, compositions of the present invention can contain one or more antisense compounds, particularly oligonucleotides, targeted to a first nucleic acid and one or more additional antisense compounds targeted to a second nucleic acid target. Alternatively, compositions of the present invention can contain two or more antisense compounds targeted to different regions of the same nucleic acid target. Two or more combined compounds may be administered together or sequentially.

In other combinations, it is contemplated that particularly where methods of treatment are contemplated, the shRNA compositions of the invention are used in combination with other anti-cancer intervention, such as combination of chemotherapy or surgery and radiation. It is contemplated that the shRNA compositions of the invention may be combined with, e.g., additional therapeutic agents, which could be normally administered to treat that condition, may also be present in the compositions of this invention. In other words, compounds of this invention can be administered as the sole pharmaceutical agent or in combination with one or more other additional therapeutic (pharmaceutical) agents where the combination causes no unacceptable adverse effects. This may be of particular relevance for the treatment of cancer. In this instance, the compound of this invention can be combined with known cytotoxic agents, signal transduction inhibitors, or with other anti-cancer agents, as well as with admixtures and combinations thereof. As used herein, additional therapeutic agents that are normally administered to treat a particular disease, or condition, are known as “appropriate for the disease, or condition, being treated”. As used herein, “additional therapeutic agents” is meant to include chemotherapeutic agents and other anti-proliferative agents. Examples of anti-cancer agents include but are not limited to known chemotherapeutic agents include, but are not limited to, for example, other therapies or anticancer agents that may be used in combination with the inventive anticancer agents of the present invention and include surgery, radiotherapy (in but a few examples, gamma-radiation, neutron beam radiotherapy, electron beam radiotherapy, proton therapy, brachytherapy, and systemic radioactive isotopes, to name a few), endocrine therapy, taxanes (taxol, taxotere etc), platinum derivatives, biologic response modifiers (interferons, interleukins, and tumor necrosis factor (TNF), TRAIL receptor targeting agents, to name a few), hyperthermia and cryotherapy, agents to attenuate any adverse effects (e.g., antiemetics), and other approved chemotherapeutic drugs, including, but not limited to, alkylating drugs (mechlorethamine, chlorambucil, Cyclophosphamide, Melphalan, Ifosfamide), antimetabolites (Methotrexate, Pemetrexed etc), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), Cell cycle inhibitors (KSP mitotic kinesin inhibitors, CENP-E and CDK inhibitors), enzymes (Asparaginase), and hormones (Tamoxifen, Leuprolide, Flutamide, and Megestrol), Gleevec™, adriamycin, dexamethasone, and cyclophosphamide. Antiangiogenic agents (Avastin and others). Kinase inhibitors (Imatinib (Gleevec), Sutent, Nexavar, Erbitux, Herceptin, Tarceva, Iressa and others). Agents inhibiting or activating cancer pathways such as the mTOR, HIF (hypoxia induced factor) pathways and others. For a more comprehensive discussion of updated cancer therapies see, http://www.nci.nih.gov/, a list of the FDA approved oncology drugs at http://www.fda.gov/cder/cancer/druglistframe.htm, and The Merck Manual, Eighteenth Ed. 2006, the entire contents of which are hereby incorporated by reference.

The shNA compounds may be combined with cytotoxic anti-cancer agents such as asparaginase, bleomycin, carboplatin, carmustine, chlorambucil, cisplatin, colaspase, cyclophosphamide, cytarabine, dacarbazine, dactinomycin, daunorubicin, doxorubicin (adriamycine), epirubicin, etoposide, 5-fluorouracil, hexamethylmelamine, hydroxyurea, ifosfamide, irinotecan, leucovorin, lomustine, mechlorethamine, 6-mercaptopurine, mesna, methotrexate, mitomycin C, mitoxantrone, prednisolone, prednisone, procarbazine, raloxifen, streptozocin, tamoxifen, thioguanine, topotecan, vinblastine, vincristine, and vindesine.

Other antineoplastic agents include e.g., aminoglutethimide, L-asparaginase, azathioprine, 5-azacytidine cladribine, busulfan, diethylstilbestrol, 2′,2′-difluorodeoxycytidine, docetaxel, erythrohydroxynonyladenine, ethinyl estradiol, 5-fluorodeoxyuridine, 5-fluorodeoxyuridine monophosphate, fludarabine phosphate, fluoxymesterone, flutamide, hydroxyprogesterone caproate, idarubicin, interferon, medroxyprogesterone acetate, megestrol acetate, melphalan, mitotane, paclitaxel, pentostatin, N-phosphonoacetyl-L-aspartate (PALA), plicamycin, semustine, teniposide, testosterone propionate, thiotepa, trimethylmelamine, uridine, vinorelbine, oxaliplatin, gemcitabine, capecitabine, epothilone and its natural or synthetic derivatives, temozolomide (Quinn et al., J. Clin. Oncology 2003, 21(4), 646-651), tositumomab (Bexxar), trabedectin (Vidal et al., Proceedings of the American Society for Clinical Oncology 2004, 23, abstract 3181), and the inhibitors of the kinesin spindle protein Eg5 (Wood et al., Curr. Opin. Pharmacol. 2001, 1, 370-377).

RNA interference (RNAi), a post-transcriptional process triggered by the introduction of double-stranded RNA, leads to gene silencing in a sequence-specific manner. Specific gene silencing of DKK-1 may be achieved in a variety of cell systems using chemically synthesized or in vitro transcribed small interfering RNA (siRNA) as well as PCR or DNA vector-based short hairpin RNA (shRNA). A few promoters have been reported to drive shRNA expression in cells, including RNA polymerase III-based promoters, U6 and H1, and RNA polymerase II promoter, CMV. In addition, the shRNA molecules of the invention may be operably linked to a prostate cell-specific promoter to achieve prostate cell specific targeting of the DKK-1 shRNA molecules to achieve silencing of the DKK-1 specifically in the prostate cells.

The present invention provides methods of gene silencing by RNA interference with DKK-1. That is, the invention can eliminate or reduce the expression of DKK-1, preferably, specifically in prostate specific tissues or cells in vitro and in vivo. The method involves creating constructs that encode the interfering (silencing) RNA in which a promoter that is active in the cell type to which the construct is going to be delivered (e.g., prostate cells) is used to drive the expression of the DKK-1 shRNA. Thus, when the construct under the control of a prostate specific promoter for example, is administered to an individual, even though the construct may enter many different types of cells in the individual, the RNA will be produced only in the one type of cell in which the promoter is active.

The methods of the present invention involve the silencing of a specific gene (a “gene of interest” or “targeted gene” or “selected gene”). By “silencing” a gene, we mean that expression of the gene product is reduced or eliminated, in comparison to a corresponding control gene that is not being silenced. Those of skill in the art are familiar with the concept of comparing results obtained with control vs. experimental results. Without being bound by theory, it is believed that RNAi is characterized by specific mRNA degradation after the introduction of homologous double stranded RNA (dsRNA) into cells. The dsRNA is recognized and processed into small interfering RNAs (siRNAs) of 19-25 nucleotides in length by an endonuclease enzyme dimer termed Dicer (RNase III family). These siRNAs, in turn, target homologous RNA for degradation by recruiting the protein complex, RNA-induced silencing complex (RISC). The complex recognizes and cleaves the corresponding mRNA (Dykxhoom D M, Novina C D and Sharp P A, Nature Review, 4: 457-467, 2003; Mittal V, Nature Reviews, 5: 355-365, 2004).

In the shRNA compositions and constructs used in the methods of the invention, there are small stretches of nucleotides that are directed against DKK-1 and are for use in modulating the expression of nucleic acid molecules encoding DKK-1. Inhibition of a DKK-1 is accomplished by providing oligomeric compounds which hybridize with one or more target nucleic acid molecules encoding DKK-1. As used herein, the terms “target nucleic acid” and “nucleic acid molecule encoding DKK-1” have been used for convenience to encompass DNA encoding DKK-1, RNA (including pre-mRNA and mRNA or portions thereof) transcribed from such DNA, and also cDNA derived from such RNA.

The principle behind antisense technology is that an antisense compound, which hybridizes to a target nucleic acid, modulates gene expression activities such as transcription or translation. This sequence specificity makes antisense compounds extremely attractive as tools for target validation and gene functionalization, as well as therapeutics to selectively modulate the expression of genes involved in disease.

Thus, the mechanism of action for the present invention involves the hybridization of the s RNA compounds with the target DKK-1 nucleic acid, wherein the outcome or effect of the hybridization is either target degradation or target occupancy with concomitant stalling of the cellular machinery involving, for example, transcription or splicing of DKK-1. Target degradation can include an RNase H. RNase H is a cellular endonuclease which cleaves the RNA strand of an RNA:DNA duplex. It is known in the art that single-stranded antisense compounds which are “DNA-like” elicit RNAse H. Activation of RNase H, therefore, results in cleavage of the RNA target, thereby greatly enhancing the efficiency of DNA-like oligonucleotide-mediated inhibition of gene expression.

Target degradation can include RNA interference (RNAi). In many species the introduction of double-stranded structures, such as double-stranded RNA (dsRNA) molecules, has been shown to induce potent and specific antisense-mediated reduction of the function of a gene or its associated gene products. The RNAi compounds are often referred to as short interfering RNAs or siRNAs. Recently, it has been shown that it is, in fact, the single-stranded RNA oligomers of antisense polarity of the siRNAs which are the potent inducers of RNAi (Tijsterman et al., Science, 2002, 295, 694-697).

Both RNAi compounds (i.e., single- or double-stranded RNA or RNA-like compounds) and single-stranded RNase H-dependent antisense compounds bind to their RNA target by base pairing (i.e., hybridization) and induce site-specific cleavage of the target RNA by specific RNAses; i.e., both are antisense mechanisms (Vickers et al., 2003, J. Biol. Chem., 278, 7108-7118). Double-stranded ribonucleases (dsRNases) such as those in the RNase III and ribonuclease L family of enzymes also play a role in RNA target degradation. Double-stranded ribonucleases and oligomeric compounds that trigger them are further described in U.S. Pat. Nos. 5,898,031 and 6,107,094.

Nonlimiting examples of an occupancy-based antisense mechanism whereby antisense compounds hybridize yet do not elicit cleavage of the target include inhibition of translation, modulation of splicing, modulation of poly(A) site selection and disruption of regulatory RNA structure. A method of controlling the behavior of a cell through modulation of the processing of an mRNA target by contacting the cell with an antisense compound acting via a non-cleavage event is disclosed in U.S. Pat. No. 6,210,892 and U.S. Pre-Grant Publication 20020049173.

Certain types of antisense compounds which specifically hybridize to the 5′ cap region of their target mRNA can interfere with translation of the target mRNA into protein. Such oligomers include peptide-nucleic acid (PNA) oligomers, morpholino oligomers and oligonucleosides (such as those having an MMI or amide internucleoside linkage) and oligonucleotides having modifications at the 2′ position of the sugar when such oligomers are targeted to the 5′ cap region of their target mRNA. This is believed to occur via interference with ribosome assembly on the target mRNA. Methods for inhibiting the translation of a selected capped target mRNA by contacting target mRNA with an antisense compound are disclosed in U.S. Pat. No. 5,789,573.

Antisense compounds targeted to a specific poly(A) site of mRNA can be used to modulate the populations of alternatively polyadenylated transcripts. In addition, antisense compounds can be used to disrupt RNA regulatory structure thereby affecting, for example, the stability of the targeted RNA and its subsequent expression.

The term “oligomeric compound” refers to a polymeric structure capable of hybridizing to a region of a nucleic acid molecule. This term includes oligonucleotides, oligonucleosides, oligonucleotide analogs, oligonucleotide mimetics and chimeric combinations of these. Oligomeric compounds are routinely prepared linearly but can be joined or otherwise prepared to be circular. Moreover, branched structures are known in the art. An “antisense compound” or “antisense oligomeric compound” refers to an oligomeric compound that is at least partially complementary to the region of a nucleic acid molecule to which it hybridizes and which modulates (increases or decreases) its expression. Consequently, while all antisense compounds can be said to be oligomeric compounds, not all oligomeric compounds are antisense compounds. An “antisense oligonucleotide” is an antisense compound that is a nucleic acid-based oligomer. An antisense oligonucleotide can be chemically modified. Nonlimiting examples of oligomeric compounds include primers, probes, antisense compounds, antisense oligonucleotides, external guide sequence (EGS) oligonucleotides, alternate splicers, and siRNAs. As such, these compounds can be introduced in the form of single-stranded, double-stranded, circular, branched or hairpins and can contain structural elements such as internal or terminal bulges or loops. Oligomeric double-stranded compounds can be two strands hybridized to form double-stranded compounds or a single strand with sufficient self complementarity to allow for hybridization and formation of a fully or partially double-stranded compound.

In one preferred embodiment of the invention, double-stranded antisense compounds encompass short interfering RNAs (siRNAs). As used herein, the term “siRNA” is defined as a double-stranded compound having a first and second strand and comprises a central complementary portion between said first and second strands and terminal portions that are optionally complementary between said first and second strands or with the target mRNA. The ends of the strands may be modified by the addition of one or more natural or modified nucleobases to form an overhang. In one nonlimiting example, the first strand of the siRNA is antisense to the target nucleic acid, while the second strand is complementary to the first strand. Once the antisense strand is designed to target a particular nucleic acid target, the sense strand of the siRNA can then be designed and synthesized as the complement of the antisense strand and either strand may contain modifications or additions to either terminus. For example, in one embodiment, both strands of the siRNA duplex would be complementary over the central nucleobases, each having overhangs at one or both termini. It is possible for one end of a duplex to be blunt and the other to have overhanging nucleobases. In one embodiment, the number of overhanging nucleobases is from 1 to 6 on the 3′ end of each strand of the duplex. In another embodiment, the number of overhanging nucleobases is from 1 to 6 on the 3′ end of only one strand of the duplex. In a further embodiment, the number of overhanging nucleobases is from 1 to 6 on one or both 5′ ends of the duplexed strands. In another embodiment, the number of overhanging nucleobases is zero.

In one embodiment of the invention, double-stranded antisense compounds are canonical siRNAs. As used herein, the term “canonical siRNA” is defined as a double-stranded oligomeric compound having a first strand and a second strand each strand being 21 nucleobases in length with the strands being complementary over 19 nucleobases and having on each 3′ termini of each strand a deoxy thymidine dimer (dTdT) which in the double-stranded compound acts as a 3′ overhang.

Each strand of the siRNA duplex may be from about 8 to about 80 nucleobases, 10 to 50, 13 to 80, 13 to 50, 13 to 30, 13 to 24, 19 to 23, 20 to 80, 20 to 50, 20 to 30, or 20 to 24 nucleobases. The central complementary portion may be from about 8 to about 80 nucleobases in length, 10 to 50, 13 to 80, 13 to 50, 13 to 30, 13 to 24, 19 to 23, 20 to 80, 20 to 50, 20 to 30, or 20 to 24 nucleobases. The terminal portions can be from 1 to 6 nucleobases. The siRNAs may also have no terminal portions. The two strands of an siRNA can be linked internally leaving free 3′ or 5′ termini or can be linked to form a continuous hairpin structure or loop. The hairpin structure may contain an overhang on either the 5′ or 3′ terminus producing an extension of single-stranded character.

In another embodiment, the double-stranded antisense compounds are blunt-ended siRNAs. As used herein the term “blunt-ended siRNA” is defined as an siRNA having no terminal overhangs. That is, at least one end of the double-stranded compound is blunt. siRNAs whether canonical or blunt act to elicit dsRNAse enzymes and trigger the recruitment or activation of the RNAi antisense mechanism. In a further embodiment, single-stranded RNAi (ssRNAi) compounds that act via the RNAi antisense mechanism are contemplated.

Further modifications can be made to the double-stranded compounds and may include conjugate groups attached to one of the termini, selected nucleobase positions, sugar positions or to one of the internucleoside linkages. Alternatively, the two strands can be linked via a non-nucleic acid moiety or linker group. When formed from only one strand, the compounds can take the form of a self-complementary hairpin-type molecule that doubles back on itself to form a duplex. Thus, the compounds can be fully or partially double-stranded. When formed from two strands, or a single strand that takes the form of a self-complementary hairpin-type molecule doubled back on itself to form a duplex, the two strands (or duplex-forming regions of a single strand) are complementary when they base pair in Watson-Crick fashion.

The oligomeric compounds in accordance with this invention may comprise a complementary oligomeric compound from about 8 to about 80 nucleobases (i.e. from about 8 to about 80 linked nucleosides). In other words, a single-stranded compound of the invention comprises from 8 to about 80 nucleobases, and a double-stranded antisense compound of the invention (such as a siRNA, for example) comprises two strands, each of which is from about 8 to about 80 nucleobases. Contained within the oligomeric compounds of the invention (whether single or double stranded and on at least one strand) are antisense portions. The “antisense portion” is that part of the oligomeric compound that is designed to work by one of the aforementioned antisense mechanisms. One of ordinary skill in the art will appreciate that this comprehends antisense portions of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleobases.

In one embodiment, the antisense compounds of the invention have antisense portions of 10 to 50 nucleobases. One having ordinary skill in the art will appreciate that this embodies antisense compounds having antisense portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleobases.

In one embodiment, the antisense compounds of the invention have antisense portions of 13 to 80 nucleobases. One having ordinary skill in the art will appreciate that this embodies antisense compounds having antisense portions of 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleobases.

In one embodiment, the antisense compounds of the invention have antisense portions of 13 to 50 nucleobases. One having ordinary skill in the art will appreciate that this embodies antisense compounds having antisense portions of 13, 14, 15, 16, 17, 18, 19, 20, 21, 22, 23, 24, 25, 26, 27, 28, 29 30, 31, 32, 33, 34, 35, 36, 37, 38, 39, 40, 41, 42, 43, 44, 45, 46, 47, 48, 49, or 50 nucleobases.

In one embodiment, the antisense compounds of the invention have antisense portions of 13 to 30 nucleobases. One having ordinary skill in the art will appreciate that this embodies antisense compounds having antisense portions of 13, 14, 15, 16, 17, 18, 19, 20, 21, 22, 23, 24, 25, 26, 27, 28, 29 or 30 nucleobases.

In some embodiments, the antisense compounds of the invention have antisense portions of 13 to 24 nucleobases. One having ordinary skill in the art will appreciate that this embodies antisense compounds having antisense portions of 13, 14, 15, 16, 17, 18, 19, 20, 21, 22, 23 or 24 nucleobases.

In one embodiment, the antisense compounds of the invention have antisense portions of 19 to 23 nucleobases. One having ordinary skill in the art will appreciate that this embodies antisense compounds having antisense portions of 19, 20, 21, 22 or 23 nucleobases.

In one embodiment, the antisense compounds of the invention have antisense portions of 20 to 80 nucleobases. One having ordinary skill in the art will appreciate that this embodies antisense compounds having antisense portions of 20, 21, 22, 23, 24, 25, 26, 27, 28, 29 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleobases.

In one embodiment, the antisense compounds of the invention have antisense portions of 20 to 50 nucleobases. One having ordinary skill in the art will appreciate that this embodies antisense compounds having antisense portions of 20, 21, 22, 23, 24, 25, 26, 27, 28, 29 30, 31, 32, 33, 34, 35, 36, 37, 38, 39, 40, 41, 42, 43, 44, 45, 46, 47, 48, 49, or 50 nucleobases.

In one embodiment, the antisense compounds of the invention have antisense portions of 20 to 30 nucleobases. One having ordinary skill in the art will appreciate that this embodies antisense compounds having antisense portions of 20, 21, 22, 23, 24, 25, 26, 27, 28, 29, or 30 nucleobases.

In one embodiment, the antisense compounds of the invention have antisense portions of 20 to 24 nucleobases. One having ordinary skill in the art will appreciate that this embodies antisense compounds having antisense portions of 20, 21, 22, 23, or 24 nucleobases.

Thus, the antisense compounds of the invention have antisense portions of 20 nucleobases or the antisense compounds of the invention have antisense portions of 19 nucleobases, or the antisense compounds of the invention have antisense portions of 18 nucleobases or the antisense compounds of the invention have antisense portions of 17 nucleobases or the antisense compounds of the invention have antisense portions of 16 nucleobases or the antisense compounds of the invention have antisense portions of 15 nucleobases or the antisense compounds of the invention have antisense portions of 14 nucleobases, or the antisense compounds of the invention have antisense portions of 13 nucleobases.

Antisense compounds 8-80 nucleobases in length comprising a stretch of at least eight (8) consecutive nucleobases selected from within the illustrative antisense compounds are considered to be suitable antisense compounds as well.

As is known in the art, a nucleoside is a base-sugar combination. The base portion of the nucleoside is normally a heterocyclic base (sometimes referred to as a “nucleobase” or simply a “base”). The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound. In addition, linear compounds may have internal nucleobase complementarity and may therefore fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

Specific examples of oligomeric compounds useful of the present invention include oligonucleotides containing modified e.g. non-naturally occurring internucleoside linkages. As defined in this specification, oligonucleotides having modified internucleoside linkages include internucleoside linkages that retain a phosphorus atom and internucleoside linkages that do not have a phosphorus atom. For the purposes of this specification, and as sometimes referenced in the art, modified oligonucleotides that do not have a phosphorus atom in their internucleoside backbone can also be considered to be oligonucleosides.

Oligomeric compounds can have one or more modified internucleoside linkages. Modified oligonucleotide backbones containing a phosphorus atom therein include, for example, phosphorothioates, chiral phosphorothioates, phosphorodithioates, phosphotriesters, aminoalkyl-phosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thiono-alkylphosphonates, thionoalkylphosphotriesters, phosphonoacetate and thiophosphonoacetate (see Sheehan et al., Nucleic Acids Research, 2003, 31(14), 4109-4118 and Dellinger et al., J. Am. Chem. Soc., 2003, 125, 940-950), selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

N3′-P5′-phosphoramidates have been reported to exhibit both a high affinity towards a complementary RNA strand and nuclease resistance (Gryaznov et al., J. Am. Chem. Soc., 1994, 116, 3143-3144). N3′-P5′-phosphoramidates have been studied with some success in vivo to specifically down regulate the expression of the c-myc gene (Skorski et al., Proc. Natl. Acad. Sci., 1997, 94, 3966-3971; and Faira et al., Nat. Biotechnol., 2001, 19, 40-44).

In some embodiments of the invention, oligomeric compounds may have one or more phosphorothioate and/or heteroatom internucleoside linkages, in particular —CH₂—NH—O—CH₂—, a methylene (methylimino) or MMI backbone, —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—). The MMI type internucleoside linkages are disclosed in the above referenced U.S. Pat. No. 5,489,677. Amide internucleoside linkages are disclosed in the above referenced U.S. Pat. No. 5,602,240.

Some oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Oligomeric compounds may also contain one or more substituted sugar moieties. Suitable compounds can comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Also suitable are O(CH₂)ₙO)ₘCH₃, O(CH₂)ₙOCH₃, O(CH₂)ₙNH₂, O(CH₂)ₙCH₃, O(CH₂)ₙONH₂, and O(CH₂)ₙON((CH₂)ₙCH₃)₂, where n and m are from 1 to about 10. Other oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. One modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—(CH₂—O—(CH₂)₂—N(CH₃)₂.

Other modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. One 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Antisense compounds may also have sugar mimetics such as cyclobutyl moieties in place of the pentofuranosyl sugar.

Oligomeric compounds can also include nucleobase (often referred to in the art as heterocyclic base or simply as “base”) modifications or substitutions. As used herein, “unmodified” or “natural” nucleobases include the purine bases adenine (A) and guanine (G), and the pyrimidine bases thymine (T), cytosine (C) and uracil (U). A “substitution” is the replacement of an unmodified or natural base with another unmodified or natural base. “Modified” nucleobases mean other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C.ident.C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-b)(1,4)benzoxazin-2(3H)-one), carbazole cytidine (2H-pyrimido(4,5-b)indol-2-one), pyridoindole cytidine (H-pyrido(3′,2′:4,5)pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al, Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are known to those skilled in ther art as suitable for increasing the binding affinity of the compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently suitable base substitutions, even more particularly when combined with 2′-O-methoxyethyl sugar modifications. It is understood in the art that modification of the base does not entail such chemical modifications as to produce substitutions in a nucleic acid sequence.

Oligomeric compounds of the present invention can also include polycyclic heterocyclic compounds in place of one or more of the naturally-occurring heterocyclic base moieties. A number of tricyclic heterocyclic compounds have been previously reported. These compounds are routinely used in antisense applications to increase the binding properties of the modified strand to a target strand. The most studied modifications are targeted to guanosines hence they have been termed G-clamps or cytidine analogs. Representative cytosine analogs that make 3 hydrogen bonds with a guanosine in a second strand include 1,3-diazaphenoxazine-2-one (Kurchavov, et al., Nucleosides and Nucleotides, 1997, 16, 1837-1846), 1,3-diazaphenothiazine-2-one, (Lin, K. -Y.; Jones, R. J.; Matteucci, M. J. Am. Chem. Soc. 1995, 117, 3873-3874) and 6,7,8,9-tetrafluoro-1,3-diazaphenoxazine-2-one (Wang, J.; Lin, K. -Y., Matteucci, M. Tetrahedron Lett. 1998, 39, 8385-8388). Incorporated into oligonucleotides these base modifications were shown to hybridize with complementary guanine and the latter was also shown to hybridize with adenine and to enhance helical thermal stability by extended stacking interactions (also see U.S. Pre-Grant Publications 20030207804 and 20030175906).

Further helix-stabilizing properties have been observed when a cytosine analog/substitute has an aminoethoxy moiety attached to the rigid 1,3-diazaphenoxazine-2-one scaffold (Lin, K. -Y.; Matteucci, M. J. Am. Chem. Soc. 1998, 120, 8531-8532). Binding studies demonstrated that a single incorporation could substantially enhance the binding affinity of a model oligonucleotide to its complementary target DNA or RNA relative to 5-methyl cytosine. On the other hand, the gain in helical stability does not compromise the specificity of the oligonucleotides.

The enhanced binding affinity of the phenoxazine derivatives together with their uncompromised sequence specificity makes them valuable nucleobase analogs for the development of more potent antisense-based drugs. Data from in vitro experiments demonstrate that heptanucleotides containing phenoxazine substitutions are capable of activating RNase H, enhancing cellular uptake and exhibit an increased antisense activity (Lin, K -Y; Matteucci, M. J. Am. Chem. Soc. 1998, 120, 8531-8532). Further activity enhancement was seen where a single substitution was shown to significantly improve the in vitro potency of a 20-mer 2′-deoxyphosphorothioate oligonucleotides (Flanagan, W. M.; Wolf, J. J.; Olson, P.; Grant, D.; Lin, K. -Y.; Wagner, R. W.; Matteucci, M. Proc. Natl. Acad. Sci. USA, 1999, 96, 3513-3518).

Another modification of the oligomeric compounds of the invention involves chemically linking to the oligomeric compound one or more moieties or conjugates which enhance the properties of the oligomeric compound, such as to enhance the activity, cellular distribution or cellular uptake of the oligomeric compound. These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmaco-dynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmaco-kinetic properties, in the context of this invention, include groups that improve uptake, distribution, metabolism or excretion of the compounds of the present invention. Representative conjugate groups are disclosed in International Patent Application PCT/US92/09196, filed Oct. 23, 1992, and U.S. Pat. Nos. 6,287,860 and 6,762,169.

Conjugate moieties include but are not limited to lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethyl-ammonium 1,2-di-O-hexadecyl-rac-gly-cero-3-H-phosphonate, a polyamine or a polyethylene glycol chain, or adamantane acetic acid, a palmityl moiety, or an octadecylamine or hexylamino-carbonyl-oxycholesterol moiety. Oligomeric compounds of the invention may also be conjugated to drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodo-benzoic acid, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic. Oligonucleotide-drug conjugates and their preparation are described in U.S. Pat. No. 6,656,730.

Oligomeric compounds can also be modified to have one or more stabilizing groups that are generally attached to one or both termini of an oligomeric compound to enhance properties such as for example nuclease stability. Included in stabilizing groups are cap structures. By “cap structure or terminal cap moiety” is meant chemical modifications, which have been incorporated at either terminus of oligonucleotides (see for example Wincott et al., WO 97/26270). These terminal modifications protect the oligomeric compounds having terminal nucleic acid molecules from exonuclease degradation, and can improve delivery and/or localization within a cell. The cap can be present at either the 5′-terminus (5′-cap) or at the 3′-terminus (3′-cap) or can be present on both termini of a single strand, or one or more termini of both strands of a double-stranded compound. This cap structure is not to be confused with the inverted methylguanosine “5′cap” present at the 5′ end of native mRNA molecules. In non-limiting examples, the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl riucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270). For siRNA constructs, the 5′ end (5′ cap) is commonly but not limited to 5′-hydroxyl or 5′-phosphate.

Particularly suitable 3′-cap structures include, for example 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate;
1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties.

It is not necessary for all positions in a given oligomeric compound to be uniformly modified, and in fact more than one of the aforementioned modifications may be incorporated in a single compound or even within a single nucleoside within an oligomeric compound.

The present invention also includes oligomeric compounds which are chimeric compounds. “Chimeric” oligomeric compounds or “chimeras,” in the context of this invention, are single- or double-stranded oligomeric compounds, such as oligonucleotides, which contain two or more chemically distinct regions, each comprising at least one monomer unit, i.e., a nucleotide in the case of an oligonucleotide compound. Chimeric antisense oligonucleotides are one form of oligomeric compound. These oligonucleotides typically contain at least one region which is modified so as to confer upon the oligonucleotide increased resistance to nuclease degradation, increased cellular uptake, alteration of charge, increased stability and/or increased binding affinity for the target nucleic acid. An additional region of the oligonucleotide may serve as a substrate for RNAses or other enzymes. By way of example, RNAse H is a cellular endonuclease which cleaves the RNA strand of an RNA:DNA duplex. Activation of RNase H, therefore, results in cleavage of the RNA target when bound by a DNA-like oligomeric compound, thereby greatly enhancing the efficiency of oligonucleotide-mediated inhibition of gene expression. The cleavage of RNA:RNA hybrids can, in like fashion, be accomplished through the actions of endoribonucleases, such as RNase III or RNAseL which cleaves both cellular and viral RNA. Cleavage products of the RNA target can be routinely detected by gel electrophoresis and, if necessary, associated nucleic acid hybridization techniques known in the art.

Chimeric oligomeric compounds of the invention can be formed as composite structures of two or more oligonucleotides, modified oligonucleotides, oligonucleosides, oligonucleotide mimetics, or regions or portions thereof. Such compounds have also been referred to in the art as hybrids or gapmers. A “gapmer” is defined as an oligomeric compound, generally an oligonucleotide, having a 2′-deoxyoligonucleotide region flanked by non-deoxyoligonucleotide segments. The central region is referred to as the “gap.” The flanking segments are referred to as “wings.” While not wishing to be bound by theory, the gap of the gapmer presents a substrate recognizable by RNase H when bound to the RNA target whereas the wings do not provide such a substrate but can confer other properties such as contributing to duplex stability or advantageous pharmacokinetic effects. Each wing can be one or more non-deoxyoligonucleotide monomers (if one of the wings has zero non-deoxyoligonucleotide monomers, a “hemimer” is described). In one embodiment, the gapmer is a ten deoxynucleotide gap flanked by five non-deoxynucleotide wings. This is referred to as a 5-10-5 gapmer. Other configurations are readily recognized by those skilled in the art. In one embodiment the wings comprise 2′-MOE modified nucleotides. In another embodiment the gapmer has a phosphorothioate backbone. In another embodiment the gapmer has 2′-MOE wings and a phosphorothioate backbone. Other suitable modifications are readily recognizable by those skilled in the art.

Oligomerization of modified and unmodified nucleosides can be routinely performed according to literature procedures for DNA (Protocols for Oligonucleotides and Analogs, Ed. Agrawal (1993), Humana Press) and/or RNA (Scaringe, Methods (2001), 23, 206-217. Gait et al., Applications of Chemically synthesized RNA in RNA: Protein Interactions, Ed. Smith (1998), 1-36. Gallo et al., Tetrahedron (2001), 57, 5707-5713).

Oligomeric compounds of the present invention can be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

Phosphorothioate-containing oligonucleotides can be synthesized by methods routine to those skilled in the art (see, for example, Protocols for Oligonucleotides and Analogs, Ed. Agrawal (1993), Humana Press). Phosphinate oligonucleotides can be prepared as described in U.S. Pat. No. 5,508,270. Other patents describing synthesis of oligonucleotides include but are not limited to U.S. Pat. Nos. 4,469,863; 5,610,289, 5,625,050; 5,256,775; 5,366,878; published PCT applications PCT/US94/00902 and PCT/US93/06976 (published as WO 94/17093 and WO 94/02499, respectively) U.S. Pat. Nos. 5,476,925; 5,023,243; 5,130,302; 5,639,873 and 5,177,198.

In general, the compositions are used in order to treat prostate cancer by reducing or eliminating the DKK-1 expression or activity in prostate cancer cells. “Reducing or eliminating” refers to a reduction or elimination of detectable amounts of the DKK-1 gene product by an amount in the range of at least about 10% to about 100%, or preferably of at least about 25% to 100%, or more preferably about 50% to about 100%, and most preferably from about 75% to about 100%. If desired, a reduction or elimination may be determined by any of several methods that are well known to those of skill in the art, and may vary from case to case, depending on the gene that is being silenced. For example, such a reduction or elimination of the expression of the gene may be determined by quantification of the gene product (e.g. by determining the quantity of a protein, polypeptide or peptide that is made) or quantification of an activity of the gene product (e.g. an activity such as enzymatic activity, signaling or transport activity, activity as a structural component of the cell, activity to change cell behaviors, activity to kill bacteria or viruses, activity to induce gene expression, etc.), or by observation and quantification of a phenotypic characteristic of the targeted cell in comparison to a control cell (e.g. lack of ability to proliferate, differentiate, or undergo apoptosis, etc). Any suitable means to determine whether or not a targeted gene has been silenced may be used. Further, the result of silencing of the gene in a cell may be highly variable, e.g. the cell may die, or become quiescent; the metabolism of the cell may be altered; the cell may lose the ability to metastasize; etc. The specific effect of silencing the gene is not a key feature of the invention, so long as the effect results in a desired outcome (e.g. ameliorating an undesired condition, or bringing about a desired condition, in the cell).

The constructs utilized in the practice of the invention include at least one cell-specific promoter that is operationally linked to nucleotides (usually DNA) encoding the desired shRNA molecule. By “operationally linked” we mean that, in the vector, the promoter is associated with the nucleotides encoding the RNA in a manner that allows the promoter to drive transcription (i.e. expression) of the RNA from the nucleotides. Transcription of RNA from, e.g. a DNA template is well-understood. Those of skill in the art will recognize that many such cell-specific promoters are known, and additional cell-specific promoters are continually being discovered. All such cell-specific promoters are encompassed by the present invention.

The promoters that are employed in the invention are cell-specific. Those of skill in the art will recognize that some tissues are made up of a single type of cell, or some types of cells are expressed only in a particular tissue, and thus, the promoter may be referred to as a “tissue-specific” promoter. In addition, some promoters may be specific for more than one, but not all, cells. These promoters may also be used in the practice of the invention, so long as it is desired to silence a gene in all cells in which the promoter is active. Examples of cell (or tissue)-specific promoters and the cells for which they are specific include but are not limited to prostate-specific antigen (PSA) promoter, prostate-specific Muc-1 promoter and the like.

The RNA molecule that is encoded by the construct of the present invention ultimately forms a double-strand RNA molecule within the cell in which it is transcribed. In general, one strand of the double-strand RNA structure will be in the range of from about 10 to about 30 ribonucleotides in length, and preferably from about 19 to about 25 ribonucleotides in length. Those of skill in the art will recognize that several viable strategies exist for forming such double-strand RNA. For example, a single RNA molecule that includes two regions that are homologus to each other and that will thus hybridize may be utilized. In this case, a hairpin loop will be formed. Alternatively, two separate RNA segments that are homologus to each other and that will thus hybridize may be formed. Other alternatives include microRNA-based hairpin RNA, etc. In one embodiment of the invention, only one gene is silenced in a particular, targeted cell type. However, this need not be the case. For example, provision of multiple constructs with the same cell-specific promoter but which encode different silencing RNAs may be used within the practice of the invention.

Further, it should be possible to express more than one silencing RNA in a single construct, driven by a single cell-specific promoter, or by more than one promoter arranged in tandem (e.g. two or more promoters). Thus, the invention contemplates using a single construct for silencing more than one gene. Alternatively, the single construct may contain multiple copies of a single promoter driving expression of two (or more) different sequences directed against DKK-1. In addition, the invention also contemplates targeting more than one cell type at a time by administering together multiple constructs that differ in targeting characteristics, i.e. constructs that differ in that they contain different cell-specific promoters. Alternatively, a single construct may contain more than one (e.g. up to four or more) cell-specific promoters operationally linked to a silencing RNA. In this case, the RNA (or RNAs) encoded by the construct will be expressed in each type of cell for which a cell-specific promoter has been included in the construct. In any case, the silencing RNAs encoded by the construct will still not be expressed in every cell that takes up the construct, but only in cells in which the cell-specific promoter is active.

In one embodiment of the invention, the promoter that is used is a constitutive promoter. However, in another embodiment, the promoter that is utilized is an inducible promoter. In this case, the formation of the silencing dsRNA in a targeted cell is not only cell specific, but expression of the RNA is activated or induced by a signal from the environment. Those of skill in the art will recognize that many suitable inducible promoters exist that could be used in the practice of the invention, examples of which include but are not limited to: (1) tetracycline-inducible system: The shRNA expression is under the control of the modified U6, H1, or 7SK promoter, in which the tetracycline operator (TetO) sequence is added. The tetracycline repressor (tTR) or tTR-KRAB expression is under the control of cell-specific promoter, such as SP-C promoter. In the absence of an inducer, the tTR or t-TR-KRAB binds to TetO and inhibits the expression of shRNA. The addition the inducer, doxycycline (DOX) removes the tTR or tTR-KRAB from the TetO and thus induces the transcription of shRNA in a cell-dependent manner since tTR or tTR-KRAB is only expressed in a specific cell type. (2) IPTG-inducible system. This is similar to (1) above except that TetO and tTR are replaced with lac operator and lac repressor, respectively. The inducer in this case is isopropyl-thio-beta-D-galactopyranoside (IPTG). (3) CER inducible system: a neomycin cassette (neo) is inserted into the U6 or H1 promoter that drives shRNA expression. The insertion disrupts the promoter activity and thus no transcription of shRNA occurs. However, the cell-specific expression of Cre recombinase under the control of a cell-specific promoter restores the promoter activity and thus the expression of shRNA in a specific cell type. The inducer in this case is tamoxifen. (4) Ecdysone-inducible system. The inducible ecdysone-responsive element/Hsmin (ERE/Hsmin) is added to U6 promoter that controls the expression of shRNA. The expression of two proteins, VgEcR and RXR are driven by cell-specific promoters. In the presence of the inducer, MurA, VgEcR and RXR form a dimer and bind to ERS/Hsmin to initiate the transcription of shRNA in a specific cell type. It will be understood that a construct can have more than one constitutive promoter, as well as combinations of constitutive and inducible promoters.

The methods of the invention involve creating constructs (e.g. vectors) that contain at least one promoter that is operationally connected to DNA that encodes RNA for silencing a specific gene. In addition, the constructs are suitable for administration to individuals that are to be treated by the methods, e.g., for treating prostate cancer. In a preferred embodiment of the present invention, the construct is an adenoviral vector for delivery as disclosed herein. However, those of skill in the art will recognize that many other systems for delivering a nucleic acid to cells already exist or are currently under development, and would be suitable for use in the practice of the present invention. For example, other vectors (both viral and non-viral) may be utilized (e.g. plasmids, viral particles, baculovirus, phage, phagemids, cosmids, phosmids, bacterial artificial chromosomes, viral DNA, P1-based artificial chromosomes, yeast plasmids, and yeast artificial chromosomes, and the like. Some forms of viral vectors may be especially useful (e.g. viral vectors such as retrovirus, lentivirus, adenovirus or adenovirus-associated vectors). Lenitviral vectors are particularly preferred. Alternatively, the construct may be delivered via liposomes or liposome-type delivery systems, or via attenuated bacterial delivery systems, by binding (either covalently or non-covalently) to another molecule which enhances delivery, by direct injection of the construct, or by catheterization, and the like. Further, other procedures which enhance the delivery of nucleic acids into cells may be utilized in conjunction with the practice of the present invention, e.g. various means of altering cell membrane permeability (e.g. ultrasound, exposure to chemicals or membrane permeability altering substances, and the like). Any appropriate means of delivery of the construct may be utilized in the practice of the present invention.

The present invention also provides a therapeutic composition comprising an effective dose of construct as described herein. The construct may conveniently be provided in the form of formulations suitable for administration to mammals. In addition, a suitable administration format may be determined by a medical practitioner for each patient individually.

In a preferred form for use by a physician, the compositions will be provided in dosage form containing an amount of a construct that will be effective in one or multiple doses to induce RNA silencing. As will be recognized by those in the field, an effective amount of therapeutic agent will vary with many factors including the age and weight of the patient, the patient's physical condition, the type of condition being treated, and other factors.

The effective dose of the constructs of this invention will typically be in the range of about 10⁷ to about 10¹² pfu (plaque forming units).

The delivery of the constructs may be in general local or systemic, and may be accomplished by a variety of methods, including but not limited to injection, positive pressure, continuous flow infusion, oral or intravenous administration, inhalation, and the like. Any suitable delivery means may be utilized in the practice of the present invention. Further, the constructs may be delivered in conjunction with other therapies.

The methods of the invention can be used to treat conditions that are caused at least in part by the expression of a particular gene. In general, conditions that are treated by the methods of the invention are those in which the phenotypic expression of the targeted gene would generally be considered unfavorable or untoward for the individual in whom the gene is expressed. For example, the expression of the gene may lead to or contribute to the development of symptoms of a disease, or may predispose an individual in whom the gene is expressed to the development of such symptoms. In specific embodiments, the compositions are used for the treatment of prostate cancer and one or more of the symptoms of prostate cancer.

Those of skill in the art will recognize that the RNAi technology of the present invention can be used to treat any condition for which it is desired to reduce or eliminate the expression of a DKK-1 gene or genes in a particular type of cell or cells.

The present invention also has useful applications as a laboratory tool. The ability to selectively silence a single gene, or specific combinations of genes, within a particular cell type allows the elucidation of the function of a specific gene (or specific combination of genes) in the cell type. The ability to do so provides a useful tool for understanding the role of specific genes in cellular metabolism, in susceptibility to disease, disease progression, or other possible functions of the gene.

The compounds of the present invention can be provided in pharmaceutical compositions by adding an effective amount of a compound to a suitable pharmaceutically acceptable diluent or carrier. Regardless of the formulation, it is expected that the compounds of the present invention will inhibit the expression of DKK-1. The compounds of the invention can also be used in the manufacture of a medicament for the treatment of prostate cancer. Suitable pharmaceutically acceptable carriers (e.g. aqueous, oil-based, etc.) and their formulation are described in standard formulations treatises, e.g., Remington's Pharmaceuticals Sciences by E. W. Martin. See also Wang, Y. J. and Hanson, M. A. “Parental Formulations of Proteins and Peptides: Stability and Stabilizers”, Journals of Parental Sciences and Technology, Technical Report No. 10, Supp. 42:2 S (1988). Constructs of the present invention should preferably be formulated in solution at neutral pH, for example, about pH 6.5 to about pH 8.5, more preferably from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, more preferably from 0.15% to 0.4% metacresol. The desired isotonicity may be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is preferred particularly for buffers containing sodium ions. If desired, solutions of the above compositions may also be prepared to enhance shelf life and stability. The therapeutically useful compositions for use in the practice of the invention are prepared by mixing the ingredients following generally accepted procedures. For example, the selected components may be mixed to produce a concentrated mixture which may then be adjusted to the final concentration and viscosity by the addition of water and/or a buffer to control pH or an additional solute to control tonicity.

Methods whereby bodily fluids, organs or tissues are contacted with an effective amount of one or more of the antisense compounds or compositions of the invention are also contemplated. Bodily fluids, organs or tissues can be contacted with one or more of the compounds of the invention resulting in modulation of DKK-1 expression in the cells of bodily fluids, organs or tissues. An effective amount can be determined by monitoring the inhibitory effect of the antisense compound or compounds or compositions on the DKK-1 target nucleic acids or their products by methods routine to the skilled artisan. Further contemplated are ex vivo methods of treatment whereby cells or tissues are isolated from a subject, contacted with an effective amount of the antisense compound or compounds or compositions and reintroduced into the subject by routine methods known to those skilled in the art.

Further contemplated herein is a method for the treatment of a subject suspected of having or at risk of having prostate cancer comprising administering to the subject an effective amount of an isolated single stranded RNA or double stranded RNA oligonucleotide directed to DKK-1. The ssRNA or dsRNA oligonucleotide may be modified or unmodified. That is, the present invention provides for the use of an isolated shRNA stranded RNA oligonucleotide targeted to DKK-1, or a pharmaceutical composition thereof, for the treatment of a disease or disorder.

The oligomeric compounds of the present invention comprise any pharmaceutically acceptable salts, esters, or salts of such esters, or any other functional chemical equivalent which, upon administration to an animal including a human, is capable of providing (directly or indirectly) the biologically active metabolite or residue thereof. Accordingly, for example, the disclosure is also drawn to prodrugs and pharmaceutically acceptable salts of the oligomeric compounds of the present invention, pharmaceutically acceptable salts of such prod rugs, and other bioequivalents.

The term “prodrug” indicates a therapeutic agent that is prepared in an inactive or less active form that is converted to an active form (i.e., drug) within the body or cells thereof by the action of endogenous enzymes or other chemicals and/or conditions. In particular, prodrug versions of the oligonucleotides of the invention are prepared as SATE ((S-acetyl-2-thioethyl) phosphate) derivatives according to the methods disclosed in WO 93/24510 or WO 94/26764.

The term “pharmaceutically acceptable salts” refers to physiologically and pharmaceutically acceptable salts of the compounds of the invention: i.e., salts that retain the desired biological activity of the parent compound and do not impart undesired toxicological effects thereto.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. of Pharma Sci., 1977, 66, 1-19). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Acid salts are the hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 22 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfoc acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For oligonucleotides, examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine. Sodium salts of antisense oligonucleotides are useful and are well accepted for therapeutic administration to humans. In another embodiment, sodium salts of dsRNA compounds are also provided.

The compositions of the invention also can be mixed with, encapsulated, conjugated or otherwise associated with other molecules, molecule structures or mixtures of compounds, as for example, liposomes, receptor-targeted molecules, oral, rectal, topical or other formulations, for assisting in uptake, distribution and/or absorption.

The compositions used in the methods of the invention may be administered in a number of ways depending upon whether local or systemic treatment is desired and upon the area to be treated. Administration may be topical (including but not limited to ophthalmic and to mucous membranes including vaginal and rectal delivery), pulmonary, e.g., by inhalation or insulation of powders or aerosols, including by nebulizer (intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Sites of administration are known to those skilled in the art. Oligonucleotides with at least one 2′-O-methoxyethyl modification are believed to be useful for oral administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. One of skill in the art will recognize that formulations are routinely designed according to their intended use, i.e. route of administration.

### EXAMPLES

The following examples provide certain illustrations for providing evidence of the efficacy of the s NA molecules in affecting the growth and proliferation of prostate cancer cells.

### Example 1

### General Materials and Methods for Monitoring DKK-1 Effects on Prostate Cancer Cells

PC-3 human prostate cancer cells were obtained from the American Type Culture Collection (Rockville, Md.). PC-3M and highly metastatic PC-3M-LN4 cells are isogenic clones selected in vivo for enhanced metastatic potential (Kozlowski J et al., Cancer Res 1984; 44: 3522-9.). Each cell line was maintained on plastic in RPMI 1640 supplemented with 10% fetal bovine serum (FBS), 1 mmol/L sodium pyruvate, 1× penicillin streptomycin, 0.1 mmol/L nonessential amino acids, 2 mmol/L L-glutamine, and 1× vitamin solution (Life Technologies, Grand Island, N.Y.), at 37° C. in 5% CO₂-95% air. C4-2B, an isogenic LNCaP variant capable of spontaneous metastasis to the bone following intraprostatic injection, was obtained from UroCor (Oklahoma City, Okla.). C4-2B cells were maintained in T-medium [80% DMEM/20% Ham's F12 (Life Technologies), 5 μg/mL insulin, 13.6 pg/mL triiodothyronine, 5 μg/mL transferrin, 0.25 μg/mL biotin, 25 μg/mL adenine (Sigma, St. Louis, Mo.), 1× penicillin/streptomycin, and 5% FBS]. C4-2B cells were infected with a retrovirus-expressing human DKK-1 or vector control containing the puromycin marker, and selected mass populations were maintained in T-medium supplemented with 1 μg/mL puromycin (Sigma). Murine bone marrow stromal ST-2 cells were obtained from RIKEN Cell Bank (Ibaraki, Japan) and maintained in a-MEM supplemented with 10% FBS, 1 mmol/L sodium pyruvate, 1× penicillin-streptomycin, and 2 mmol/L L-glutamine. All cells were shown free of Mycoplasma by PCR ELISA (Roche Diagnostics, Indianapolis, Ind.).

The expression of Wnt family members was evaluated in both the PC-3M and LNCaP prostate cancer cell systems. Unique primer sets for 19 Wnts, four Frizzled, two lrp, DKK-1, sFRP-5, and glyceraldehyde-3-phosphate dehydrogenase were designed using Primer3 (15) and blasted against the human genome to confirm specificity and to ensure no cross-reactivity. To do PCR, total RNA was isolated from subconfluent cells using TRIzol Reagent (Invitrogen, Carlsbad, Calif.). One microgram of total RNA/primer pair was then amplified using the access reverse transcription-PCR(RT-PCR) system per manufacturer's instructions (Promega, Madison, Wis.). PCR was done in a Perkin-Elmer GeneAmp 9700 as follows: 48° C., 45 minutes; 94° C., 5 minutes followed by 30 cycles at 94° C. for 25 seconds, 58° C. for 30 seconds, and 72° C. for 30 seconds. This was not designed to be in the linear range of the amplification process; thus, we can only make semiquantitative statements when differences are large. All products were evaluated by electrophoresis on 1.2% agarose gels.

DKK-1 RNA levels in prostate cancer cell lines were evaluated by quantitative real-time RT-PCR using the primers for DKK-1 indicated in Table 1. Briefly, 100-ng RNA was amplified using the LightCycler SYBR Green kit according to the manufacturer's instructions (Roche Diagnostics) on a LightCycler. Briefly, amplification was done at 94° C. for 5 seconds, 62° C. for 5 seconds, and 72° C. for 5 seconds for 45 cycles. RT-PCR of β-actin was used as an internal control to normalize for loading differences between samples.

TABLE 1
PCR primers used to evaluate Wnt family member aud receptor expression

| | | SEQ | | SEQ | |
|---|---|---|---|---|---|
| | | ID | | ID | Amplicon |
| Primer | Forward | NO: | Reverse | NO: | size (bp) |
| Wnt 1 | 5′-ACCCAATCCCTCTCCACTCT-3′ | 26 | 5′-GATTCAAGGAAAAGCCACCA-3′ | 27 | 214 |
| Wnt 2 | 5′-GTGGATGCAAAGGAAAGGAA-3′ | 28 | 5′-AGCCAGCATGTCCTGAGAGT-3′ | 29 | 152 |
| Wnt 2b | 5′-GTGTCCTGGCTGGTTCCTTA-3′ | 30 | 5′-AGCTGGTGCAAAGGAAAGAA-3′ | 31 | 186 |
| Wnt 3 | 5′-TGTGAGGTGAAGACCTGCTG-3′ | 32 | 5′-AAAGTTGGGGGAGTTCTCGT-3′ | 33 | 207 |
| Wnt 3a | 5′-GGACAAAGCTACCAGGGAGT-3′ | 34 | 5′-ACTCGATGTCCTCGCTACAG-3′ | 35 | 176 |
| Wnt 4 | 5′-CTCATGAACCTCCACAACAA-3′ | 36 | 5′-GCACCATCAAACTTCTCCTT-3′ | 37 | 170 |
| Wnt 5a | 5′-CTTGGTGGTCGCTAGGTATG-3′ | 38 | 5′-TCGGAATTGATACTGGCATT-3′ | 39 | 194 |
| Wnt 5b | 5′-GGGCTTTTTCTCTCCCTCTG-3′ | 40 | 5′-CGAGGTTGAAGCTGAGTTCC-3′ | 41 | 219 |
| Wnt 6 | 5′-GTCACGCAGGCCTGTTCTAT-3′ | 42 | 5′-CGTCCATAAAGAGCCTCGAC-3′ | 43 | 208 |
| Wnt 7a | 5′-TCTCATGAACTTGCACAACA-3′ | 44 | 5′-ACTTGTCCTTGAGCACGTAG-3′ | 45 | 138 |
| Wnt 7b | 5′-GCCTGCAGGTCCTAGAAGTG-3′ | 46 | 5′-CTCCCAAAGTGCTGGGATTA-3′ | 47 | 172 |
| Wnt 8a | 5′-GAACTGCCCTGAAAATGCTC-3′ | 48 | 5′-ATCCTTTCCCCAAATTCCAC-3′ | 49 | 237 |
| Wnt 8b | 5′-CCATGAACCTGCACAACAAC-3′ | 50 | 5′-TGAGTGCTGCGTGGTACTTC-3′ | 51 | 174 |
| Wnt 9a | 5′-GCAAGCATCTGAAGCACAAG-3′ | 52 | 5′-TGCTCTCGCAGTTCTTCTCA-3′ | 53 | 246 |
| Wnt 9b | 5′-GAGGACTCACCCAGCTTCTG-3′ | 54 | 5′-TAGGCCTAGTGCTTGCAGGT-3′ | 55 | 227 |
| Wnt 10a | 5′-GGTTGCTCCACACCCTAAAA-3′ | 56 | 5′-ATGATGAAGGGAATGGTGGA-3′ | 57 | 208 |
| Wnt 10b | 5′-AGTTCTCTCGGGATTTCTTG-3′ | 58 | 5′-CGCTTCAGGTTTTCAGTTAC-3′ | 59 | 113 |
| Wnt 11 | 5′-CCCAAGCCAATAAACTGATG-3′ | 60 | 5′-AGGTATCGGGTCTTGAGGTC-3′ | 61 | 230 |
| Wnt 16 | 5′-GCTCCTGTGCTGTGAAAACA-3′ | 62 | 5′-TGCATTCTCTGCCTTGTGTC-3′ | 63 | 249 |
| FZD 2 | 5′-GTCCTCAAGGTGCCATCCTA-3′ | 64 | 5′-CAGCCCGACAGAAAAATGAT-3′ | 65 | 248 |
| FZD 3 | 5′-CTCTCTTTGGCCCTTGACTG-3′ | 66 | 5′-ACAAAGAAAAGGCCGGAAAT-3′ | 67 | 223 |
| FZD 5 | 5′-TTCTGGATAGGCCTGTGGTC-3′ | 68 | 5′-CGTAGTGGATGTGGTTGTGC-3′ | 69 | 214 |
| FZD 6 | 5′-TTGTTGGCATCTCTGCTGTC-3′ | 70 | 5′-CCATGGATTTGGAAATGACC-3′ | 71 | 222 |
| Lrp 5 | 5′-GCCATCGACTATGACCCACT-3′ | 72 | 5′-CAGAACAGTGTCCGGCTGTA-3′ | 73 | 179 |
| Lrp 6 | 5′-CCCATGCACCTGGTTCTACT-3′ | 74 | 5′-CTGGAACTGGGACTCTGAGC-3′ | 75 | 192 |
| DKK-1 | 5′-TAGCACCTTGGATGGGTATT-3′ | 76 | 5′-ATCCTGAGGCACAGTCTGAT-3′ | 77 | 110 |
| sFRP-5 | 5′-GATGTGCTCCAGTGACTTTG-3′ | 78 | 5′-GCTTGAGCAGCTTCTTCTTT-3′ | 79 | 107 |
| GAPDH | 5′-CCAAGGTCATCCATGACAAC-3′ | 80 | 5′-AGAGGCAGGGATGATGTTCT-3′ | 81 | 149 |
| β-actin | 5′-GGACTTCGAGCAAGAGATGG-3′ | 82 | 5′-AGCACTGTGTTGGCGTACAG-3′ | 83 | 234 |

The ONCOMINE database and gene microarray analysis tool, a repository for published cDNA microarray data (Rhodes et al., Neoplasia (New York) 2004; 6:1-6.), was explored for mRNA expression of Wnt pathway mediators in normeoplastic prostate, primary prostate cancer, and prostate cancer metastases. Statistical analysis of differences was done using ONCOMINE algorithms to account for the multiple comparisons among different studies, similar to a meta-analysis, as previously described (Rhodes et al., Neoplasia (New York) 2004; 6:1-6).

Design of DKK-1 short hairpin RNAs. The Block-it RNAi designer (Invitrogen) was used to design a short hairpin RNA molecules (shRNA) specific to human DKK-1 (accession no. NM_{—}012242; position 351-371; 5′-CAATGGTCTGGTACTTATTCCCGAAGGAATAAGTACCAGACCATTGCACC-3′ (SEQ ID NO: 84). A DKK-1 shRNA control was generated by inverting the bases at position 9-13 within the DKK-1 351 siRNA. Resulting sequences were cloned into the RNA expression vector pENTR/H1/TO (Invitrogen) and sequence confirmed. DKK-1 shRNAs and control were transfected into human PC-3 prostate cancer cells using a CaPO₄ method and individual clones selected using 100 μg/mL Zeocin (Invitrogen).

Western blot for DKK-1 Expression. The amount of DKK-1 protein was determined using Western blotting of total cell lysates. Briefly, cells were washed once on ice with ice-cold PBS and scraped into 0.3 mL ice-cold lysis buffer [1% Triton X-100, 20 mmol/L Tris-HCl (pH 8.0), 137 mmol/L NaCl, 10% glycerol (v/v), 2 mmol/L EDTA, 1 mmol/L phenylmethylsulfonyl fluoride, 10 μg/mL aprotinin, 10 Ag/mL leupeptin, 50 Ag/mL trypsin inhibitor, 1 mmol/L sodium orthovanadate]. Cell lysates were then clarified by centrifugation and aliquots of each were removed for protein determination by the bicinchoninic acid protein assay (Pierce, Rockford, Ill.). Equal amounts of protein (30 μg/sample) were resolved using 10% SDS-PAGE. Separated proteins were transferred onto 0.45-μm polyvinylidene difluoride membranes (Millipore, Bedford, Mass.). The filters were blocked with 3% bovine serum albumin in TBS and probed with goat anti-human DKK-1:pAb (1:1,000, R&D Systems, Minneapolis, Minn.). Protein bands were visualized using the enhanced chemiluminescence detection system (Cell Signaling, Beverly, Mass.). To normalize for differences in loading, the blots were stripped and reprobed with mouse anti-h-actin monoclonal antibody (1:1,000, Sigma).

Cell proliferation. Cells were plated in 96-well plates at a density of 1.5×10³ cells per 0.2 mL per well in complete medium in triplicate. The total number of viable cells on one plate was determined every 24 hours by the addition of a final concentration of 1 mmol/L 3-(4,5-dimethyl-2-thiazolyl)-2,5-diphenyl-2H-tetrazolium bromide. The formazan product was dissolved in DMSO and absorbancies were read at 570 nm on a Spectra Max Plus plate reader (Molecular Devices, Sunnyvale, Calif.).

Cell/cell in vitro mineralization assay. Cells were trypsinized, washed, and treated in suspension with a cytostatic dose of 15 Gy of γ-radiation using a ¹³⁷Cs source. To ensure that irradiation did not alter Wnt gene expression RT-PCR was also done on PC-3 parental, shRNA control, and DKK1 shRNA-transfected cells 48 hours after 15-Gy g-radiation. Irradiation did not affect Wnt expression (data not shown). Irradiated prostate cancer cells (6.0×10⁴) were plated with or without 6.0×10⁴ ST-2 murine bone marrow stromal cells to 12-well plates. After 24 hours, cells were changed to a mineralization media (maintenance media plus 50 μg/mL ascorbic acid and 5 mmol/L h-glycerophosphate). ST-2 cells in mineralization media or with the addition of rBMP-2 (Peprotech, Rocky Hill, N.J.) were used as mineralization negative and positive controls, respectively. Separately, rWnt3a (R&D Systems) was evaluated to show Wnt-mediated mineralization. Following 11 days in culture, conditioned medium was evaluated for alkaline phosphatase activity using the Sigma Diagnostics Alkaline Phosphatase kit (Sigma). The presence of mineral was determined by staining for calcium phosphate using silver nitrate (von Kossa staining) at the experimental end point as previously described (Lin et al. Prostate 2001; 47:212-21.).

### Example 2

### In Vivo Animal Model of Bone Metastasis

The effect of prostate cancer-derived DKK-1 expression on bone turnover was evaluated following direct injection into the tibia of male C.B17 severe combined immunodeficient mice (intratibial injection) as described previously (Zhang et al. J Clin Invest 2001; 107:1235-44; Corey et al. Prostate 2002; 52:20-33). Tumors were allowed to grow for 12 weeks at which time mice were sacrificed. Evidence of tumor-induced bone change was evaluated at 12 weeks after tumor injection using Faxitron radiography (Faxitron X-ray Corp, Wheeling, Ill.). Radiographs were digitized and the percent osteolytic area of the total tibial bone area was quantified using Scion Imaging Software (Scion Corp., Fredrick, Md.). Tumor-injected tibiae and controlateral tibiae without tumors were removed, fixed in 10% normal buffered formalin, and bone mineral density (BMD) measured using dual-energy X-ray absorptiometry (DEXA) with a pDEXA Sabre scanner (Orthometrix, Inc., White Plains, N.Y.) as previously described (Zhang et al., Cancer Res 2003; 63:7883-90.). Following DEXA analysis, tibiae were decalcified in Cal-Ex II (Fisher Scientific, Hampton, N.H.), paraffin embedded, and histologic sections stained with H&E. The animal protocol was approved by the University of Michigan Institutional Animal Care and Use Committee.

Results and Discussion:

Bone is the most common metastatic site of prostate cancer (CaP). Growth of CaP within the skeleton induces extensive bone remodeling that results in the development of osseous lesions consisting of regions of both bone formation (osteosclerosis) and bone resorption (osteolysis). Wnts are cysteine-rich glycoproteins that mediate bone development in the embryo and promote bone production in the adult. It was previously shown by the inventors that blocking canonical Wnts by over-expressing the soluble Wnt inhibitor dickkopf-1 (DKK-1) transforms osteoblastic C4-2B CaP cells into a highly osteolytic tumor in vivo. These data suggest that Wnts contribute to the osteoblastic component of CaP osseous lesions and that DKK-1, by blocking Wnt activity, promotes an osteolytic environment. Since the inhibition of bone resorption decreases CaP osseous lesions in tumor bearing mice, it was tested whether blocking DKK-1 could suppress CaP bone metastasis. Through the use of DKK-1 shRNA, greater than 80% reduction in DKK-1 protein levels in human PC-3 CaP cells was achieved. When injected directly into the tibia of nude mice, PC-3 cells transfected with control shRNA formed highly osteolytic lesions with high frequency (12/13 mice). In contrast, reduction of DKK-1 using DKK-1 shRNA significantly reduced the ability of PC-3 cells to develop tumors within the bone (2/15 mice). DKK-1 suppression also reduced the incidence and size of subcutaneous tumors but not attachment dependent growth in vitro suggesting that restoration of Wnt signaling in PC-3 cells had a generalized anti-tumor effect in vivo. To confirm these findings, mice were treated systemically with a DKK-1 neutralizing antibody and the effect on metastasis following intracardiac injection of PC-3 cells was measured. DKK-1 neutralizing antibody (5 mg/kg) twice weekly decreased the overall tumor burden 8.3-fold compared to IgG-treated control mice over a period of 6 weeks. These data demonstrate that decreasing DKK-1 decreases CaP tumor burden. These results suggest that Wnt activity has an anti-tumor effect in transformed cells, as opposed to its well-recognized oncogenic effect in the transformation of normal cells to neoplastic cells. Accordingly, inhibition of Wnt inhibitors, such as DKK-1, may have therapeutic effects to diminish CaP progression.

To further investigate the effect of DKK-1 suppression of soft tissue tumor growth, PC-3 DKK-1 shRNA or control cells transfected with non-specific shRNA were injected into the subcutis of nude mice and the incidence and growth rate of the tumors measured. The in vivo growth rate or tumorigenicity of DKK-1 and control shRNA cells was measured following the injection of 1×10⁶ cells into the subcutis of male nude mice. Twice a week for the length of the study, tumor diameter was measured in two axes using a caliper. Tumor volume was then calculated using the following formula (min²× max)/2. The data show that PC-3 DKK-1 shRNA transfected cells grew as poorly in the skin as they had in the bone compared to DKK-1⁺ shRNA control cells, n=2 (tumor incidence PC-3 DKK control shRNA 7/9 (78%); PC-3 DKK shRNA c18 10/15 (67%)). Although the tumor incidences were approximately the same in each group, the DKK-1 shRNA transfected cells developed tumors at a much slower rate as reflected in decreased tumor volume and weight. Taken together, the data demonstrate that knock-down of DKK-1 with either an shRNA or neutralizing antibody reduced PCa tumor growth in vivo.

### Example 3

### DKK-1 Knock-Down Increases OPG and CDKN1A/2B Expression

The mechanism through which DKK-1 suppression leads to a reduction in tumor growth is unknown. Reductions of tumor growth within the bone can result from alterations in one or a combination of the following: 1) RankL/OPG expression, 2) apoptosis, and/or 3) cell cycle modulation. An increase in tumor cell apoptosis or a decrease in the rate of cell cycling would have a negative impact on tumor growth at any site. Within the context of the bone, β-catenin signaling in osteoblasts was shown to decrease osteoclastogenesis through the production of OPG (Glass, 2005).

To investigate each of these possibilities, basal gene expression between parental and DKK-1 shRNA cells was compared using RT2 profiler PCR arrays. The expression of OPG, p21, and p15 were evaluated in PC-3 parental, PC-3 DKK-1 and control shRNA cells by both semi-quantitative PCR and quantitative PCR. Total RNA was isolated from subconfluent cells using RNeasy RNA isolation kit (Qiagen, Valencia, Calif.). cDNA was then prepared from 1 μg of total RNA using the reverse transcription system (Promega, Madison, Wis.). To perform semi-quantitative PCR, 5 μl of RT reaction was amplified with Taq polymerase (Promega) and a final concentration of 2 mM MgCl₂, 25 pMol primers, and 1 mM dNTPs. PCR was performed in a Perkin Elmer GeneAmp 9700 as follows: 48° C., 45 minutes; 94° C., 5 minutes followed by 30 cycles at 94° C., 45 seconds; 58° C., 45 seconds; and 72° C., 60 seconds. All products were evaluated by electrophoresis on 1.2% agarose gels. For quantitative PCR, 2 μl of cDNA was then amplified using the LightCycler SYBR Green DNA master mix according to the manufacturer's instructions (Roche Diagnostics, Indianapolis, Ind.). Amplification was performed at 94° C. for 10 sec, 58° C. for 30 sec, and 72° C. for 30 sec for 45 cycles. The primers used were as follows:

| | | |
|---|---|---|
| OPG-976F | 5′ GGCAACACAGCTCACAAGAA 3′ | SEQ ID NO: 20 |
| OPG-1216R | 5′ CTGGGTTTGCATGCCTTTAT 3′ | SEQ ID NO: 21 |
| p15-1050F | 5′ GACCGGGAATAACCTTCCAT 3′ | SEQ ID NO: 22 |
| p15-1234R | 5′ CACCAGGTCCAGTCAAGGAT 3′ | SEQ ID NO: 23 |
| p21-1131F | 5′ ATGAAATTCACCCCCTTTCC 3′ | SEQ ID NO: 24 |
| p21-1304R | 5′ CCCTAGGCTGTGCTCACTTC 3′ | SEQ ID NO: 25 |

PCR of β-actin and GAPDH were used as an internal control to normalize for loading differences between samples as previously described (Hall, 2005). Analysis of DKK-1 shRNA cells showed a 12 fold increase in the RankL/TRAIL inhibitor osteoprotegerin (OPG), a 10 fold increase in Cyclin-dependent kinase inhibitor 1A (p21/Cip1/CDKN1A), and a 4 fold increase in Cyclin-dependent kinase inhibitor 2B (p15/INK4B/CDKN2B). Both semi-quantitative and quantitative PCR analysis confirmed the increase in these RNA transcripts in DKK-1 shRNA cells. Increased expression of these proteins was also demonstrated in DKK-1 shRNA cells by Western blotting. To verify that these increases were a result of restoration of canonical Wnt signaling following DKK-1 knock-down, PC-3 PCa cells were transiently transfected with a full-length β-catenin expression vector and cell lysates probed for OPG/p15/p21. An expression vector containing full-length human β-catenin was the kind gift of Eric Fearon (University of Michigan). 2.5×10⁵ PC-3 cells/6 well dish were transfected with 1 μg β-catenin expression vector using Fugene 6 according to manufacturer's instructions (Roche, Indinanpolis, Ind.). Following 48-hour incubation, whole cell lysates were prepared and subjected to western blot analysis. The amount of OPG/p15/p21 protein in DKK-1 shRNA and β-catenin transfected cells was determined using western blotting of total cell lysates as described previously (Hall, 2005). Equal amounts of protein (30 μg/sample) were resolved using 10% SDS-polyacrylamide gel electrophoresis (PAGE). Separated proteins were transferred onto 0.45 μm PVDF membranes (Millipore, Bedford, Mass.). The filters were blocked with 3% BSA in Tris-buffered saline and probed with goat anti-human OPG pAb (1:1000, R&D Systems, Minneapolis, Minn.); mouse anti-p21:mAb (1:250, BD Biosciences, San Jose, Calif.); and rabbit anti-p15 pAb (1:1000, Abcam, Cambridge, Mass.). Protein bands were visualized using the ECL detection system (Cell Signaling, Beverly, Mass.). To normalize for differences in loading, the blots were stripped and reprobed with mouse anti-β-actin mAb (1:1000, Sigma, St. Louis, Mo.). The data show that transfection with β-catenin induced the expression of OPG, p15, and p21 protein. Taken together, the data demonstrate that targeted DKK-1 knock-down leads to increases in key inhibitors of osteoclastogenesis and the cell cycle. Induction of these inhibitors through increased Wnt signaling provide possible mechanisms for decreased tumor growth following DKK-1 suppression in PC-3 PCa cells.

The data demonstrate that blocking DKK-1 using both a targeted shRNA and a neutralizing antibody reduced tumor establishment in both soft tissue and bone. Blocking DKK-1 not only increased tumor cell expression of OPG, which may block osteoclastogenesis and tumor establishment within the bone, but also increased cyclin dependent kinase inhibitors p15 and p21, which may reduce tumor growth. It has been demonstrated previously that blocking RankL through the systemic delivery of OPG reduced the establishment of PCa tumors within the bone (Zhang, 2001; Mornoy, 2001). Recently, it was shown in a bone xenograph SCID-rab model that daily injections of anti-human DKK-1 neutralizing antibody into the area surrounding the implant increased the bone mineral density of implanted rabbit long bones (Yaccaby, 2007). The bone anabolic effect of anti-DKK-1 antibody reduced tumor growth of multiple myleoma cells injected within the bone implant demonstrating that DKK-1 controls myeloma growth within the bone. The mechanism for this effect is presumably due to Wnt-mediated induction of OPG followed by reductions in osteoclast number similar to that which was observed in transgenic mice with an osteoblast specific expression of β-catenin (Glass, 2005). As osteolysis is reported to support tumor establishment within the bone via a vicious cycle (Siclari, C M R 2006), it follows that Wnt-mediated induction of OPG should suppress the formation of bone lesions.

### Example 4

### Sequences of DKK-1 Against which Particular shRNA Molecules can be Created

The following sequence is an exemplary DKK-1 sequence. Highlighted therein are four specific areas to which shRNA molecules can be directed. Given the teachings of the present invention, one of skill in the art will readily be able to prepare further shRNA molecules as well as modify the shRNA molecules illustrated below to yield further molecules that will be useful in the methods of the present invention.

```
cDNA sequence human Dickkopf-1 (DKK-1)
SEQ ID NO: 1
      1 gcagagctct gtgctccctg cagtcaggac tctgggaccg cagggggctc ccggaccctg
    61 actctgcagc cgaaccggca cggtttcgtg gggacccagg cttgcaaagt gacggtcatt
  121 ttctctttct ttctccctct tgagtccttc tgagatgatg gctctgggcg cagcgggagc
  181 tacccgggtc tttgtcgcga tggtagcggc ggctctcggc ggccaccctc tgctgggagt
  241 gagcgccacc ttgaactcgg ttctcaattc caacgctatc aagaacctgc ccccaccgct
  301 gggcggcgct gcggggcacc caggctctgc agtcagcgcc gcgccgggaa tcctgtaccc
  361 gggcgggaat aagtaccaga ccattgacaa ctaccagccg tacccgtgcg cagaggacga
  421 ggagtgcggc actgatgagt actgcgctag tcccacccgc ggaggggacg caggcgtgca
  481 aatctgtctc gcctgcagga agcgccgaaa acgctgcatg cgtcacgcta tgtgctgccc
  541 cgggaattac tgcaaaaatg gaatatgtgt gtcttctgat caaaatcatt tccgaggaga
  601 aattgaggaa accatcactg aaagctttgg taatgatcat agcaccttgg atgggtattc
  661 cagaagaacc accttgtctt caaaaatgta tcacaccaaa ggacaagaag gttctgtttg
  721 tctccggtca tcagactgtg cctcaggatt gtgttgtgct agacacttct ggtccaagat
  781 ctgtaaacct gtcctgaaag aaggtcaagt gtgtaccaag cataggagaa aaggctctca
  841 tggactagaa atattccagc gttgttactg tggagaaggt ctgtcttgcc ggatacagaa
  901 agatcaccat caagccagta attcttctag gcttcacact tgtcagagac actaaaccag
  961 ctatccaaat gcagtgaact ccttttatat aatagatgct atgaaaacct tttatgacct
1021 tcatcaactc aatcctaagg atatacaagt tctgtggttt cagttaagca ttccaataac
1081 accttccaaa aacctggagt gtaagagctt tgtttcttta tggaactccc ctgtgattgc
1141 agtaaattac tgtattgtaa attctcagtg tggcacttac ctgtaaatgc aatgaaactt
1201 ttaattattt ttctaaaggt gctgcactgc ctatttttcc tcttgttatg taaatttttg
1261 tacacattga ttgttatctt gactgacaaa tattctatat tgaactgaag taaatcattt
1321 cagcttatag ttcttaaaag cataaccctt taccccattt aattctagag tctagaacgc
1381 aaggatctct tggaatgaca aatgataggt acctaaaatg taacatgaaa atactagctt
1441 attttctgaa atgtactatc ttaatgctta aattatattt ccctttaggc tgtgatagtt
1501 tttgaaataa aatttaacat ttaatatcat gaaatgttat aagtagacat acattttggg
1561 attgtgatct tagaggtttg tgtgtgtgta cgtatgtgtg tgttctacaa gaacggaagt
1621 gtgatatgtt taaagatgat cagagaaaag acagtgtcta aatataagac aatattgatc
1681 agctctagaa taactttaaa gaaagacgtg ttctgcattg ataaactcaa atgatcatgg
1741 cagaatgaga gtgaatctta cattactact ttcaaaaata gtttccaata aattaataat
1801 acctaaaaaa aaaaa
```

From the above sequence, the exemplary sequences targeted by the shRNA were:

| | | |
|---|---|---|
| 366 | GGAATAAGTACCAGACCATTG | SEQ ID NO: 2 |
| 834 | GCTCTCATGGACTAGAAATAT | SEQ ID NO: 3 |
| 1096 | GGAGTGTAAGAGCTTTGTTTC | SEQ ID NO: 4 |
| 1383 | GGATCTCTTGGAATGACAAAT | SEQ ID NO: 5 |

Thus in specific embodiments of the invention there are provided sequences that are antisense to the above four sequences. Other sequences can be identified simply by performing a gene walk along SEQ ID NO:1 and selecting those regions that provide any type of inhibition of DKK-1 expression. The following specific shRNA molecules are 21-mers it is contemplated that the skilled artisan could perform a gene-walk along SEQ ID NO:1 and generate all possible 21-mer shRNA molecules and test them in the assays described above. In this manner the skilled artisan will readily identify additional shRNA molecules. While the preferred molecules identified are 21-mers, it is contemplated that shorter and longer shRNA molecules also will be useful.

Thus, shRNA sequences of the present invention include:

| | |
|---|---|
| CCTTATTCATGGTCTGGTAAG | (SEQ ID NO: 6) |
| CGAGAGTACCTGATCTTTATA | (SEQ ID NO: 7) |
| CCTCACATTCTCGAAACAAAG | (SEQ ID NO: 8) |
| CCTAGAGAACCTTACTGTTTA | (SEQ ID NO: 9) |

Compositions comprising combinations and variations of these sequences are also contemplated. Additional target regions from DKK-1 include:

| | |
|---|---|
| GGTTCTCAATTCCAACGCTAT | (SEQ ID NO: 10) |
| GGTAATGATCATAGCACCTTG | (SEQ ID NO: 11) |
| GCTAGACACTTCTGGTCCAAG | (SEQ ID NO: 12) |
| GCCGGATACAGAAAGATCACC | (SEQ ID NO: 13) |
| GCACTTACCTGTAAATGCAAT | (SEQ ID NO: 14) |

Thus, shRNA sequences of the present invention that target to the above sequences include:

| | |
|---|---|
| CCAAGAGTTAAGGTTGCGATA | (SEQ ID NO: 15) |
| CCATTACTAGTATCGTGGAAC | (SEQ ID NO: 16) |
| CGATCTGTGAAGACCAGGTTC | (SEQ ID NO: 17) |
| CGGCCTATGTCTTTCTAGTGG | (SEQ ID NO: 18) |
| CGTGAATGGACATTTACGTTA | (SEQ ID NO: 19) |

Compositions comprising combinations and variations of these sequences are also contemplated.

### Example 5

### DKK-1 Expression Decreases with PCa Progression

Cases of clinically localized PCa were identified from a radical prostatectomy series at the University of Michigan. PCa metastases were obtained from the Rapid Autopsy Program through the Michigan Prostate SPORE Tissue Core (Rubin, 2000). To study the expression of DKK-1 in PCa, two tissue microarrays (TMAs) were used that consisted of a total of 758 evaluable samples of non-neoplastic prostate (n=57), localized PCa (n=79) and metastatic, hormone-refractory PCa (n=55). The metastatic TMAs included PCa metastatic to the liver, lung, bone, lymph node, brain, adrenal, and soft tissue. At least six 0.6-mm cores were taken from each sample.

Tissue microarrays (TMA) prepared by the University of Michigan prostate SPORE tissue core were used to conduct a retrospective analysis of DKK-1 expression in human prostate cancer patient specimens. Two types of TMAs were evaluated. A progression TMA contained a selection of non-neoplastic prostate, PIN lesions, primary lesions, and metastases. The autopsy array was composed of soft tissue and skeletal metastases obtained from the SPORE rapid autopsy program. Together these TMAs were used to evaluate DKK-1 expression during progression and to explore the impact of organ site on DKK-1 expression. The progression TMA contained a total of 286 cores representing 72 total PCa patients. The core distribution was as follows: Non-neoplastic=92; PIN=19; Primary tumor=142; and Metastases=33. The metastases samples were comprised of PCa lymph node and liver metastases. The staining intensity and percentage of DKK-1 positive cells were determined for each core. It was observed that the Expression Index (EI), which is the product of the staining intensity and percent expression, was most representative of the data for each individual core. The data show that DKK-1 expression was restricted to epithelial cells in all samples. DKK-1 was found at low levels in normal prostate tissue but increased significantly in both PIN and primary PCa lesions. In PCa metastases, DKK-1 expression was greater than normal prostate but at significantly lower levels compared to primary PCa prostate tumors. Graphical evaluation of the total EI data for each stage demonstrated that DKK-1 expression increased 5-fold from normal prostate to primary lesions but decreased 47% from primary to metastatic lesions (normal prostate: mean, 19.3±5.6, median 0; primary PCa: mean, 106.0±10.4, median, 60; PCa metastases: mean, 56.3±21.5, median 0; p<0.008 PCa metastases vs. primary lesions). Taken together, the data demonstrate that DKK-1 expression increased during PCa development but decreased as the tumor progressed.

### Example 6

### Concomitant Decrease in Nuclear β-Catenin and DKK-1 During PCa Progression

Aberrant β-catenin signal transduction has been implicated in the development of several types of cancer including PCa (Yardy, 2005). Further, DKK-1 was recently described as a gene target of β-catenin (Gonzalez-Sancho, 2005). β-catenin expression was therefore evaluated on the PCa progression TMA and the data was related to DKK-1 expression. Formalin fixed, paraffin sections were dehydrated to buffer and antigen retrieved by pretreatment with Citrate Buffer, pH6.0 for 10 minutes and microwaving. After Peroxidase blocking, antibody: DDK-1 is stained at a dilution of 1:400 at room temperature on the DAKO AutoStainer using the LSAB+ detection kit. Chromagen is applied for 5 minutes. Counterstain is Hematoxylin. Antibody: DKK-1, Goat polyclonal (ab22827; Abcam Inc, Cambridge, Mass.). DKK-1 staining intensity was scored by a genitorurinary pathologist as negative [1], weak [2], moderate [3], or strong [4] based on the amount of stain detected. The percent of positive stained cells was determined by counting 100 cells in 2 random fields. Number of samples: Non-neoplastic=92; PIN=19; Primary tumor=142; and Metastases=33.

In addition to determining the staining intensity and percent expression of β-catenin, the distribution of β-catenin (membranous, cytoplasmic, or nuclear) was also recorded. The β-catenin antibody used in this study detected both, and can not discriminate between, wild-type and mutant β-catenin. As a result, the staining intensity was strong and percent expression was greater than 96% in normal, PIN and primary PCa lesions. The distribution in these samples was 80% membranous with the remainder in the nucleus. However, in PCa metastases, total β-catenin expression fell to 64.5% and was distributed between the membrane and the cytoplasm, 66.3% vs. 33.7%, respectively. As nuclear β-catenin levels were high in both normal and PCa primary lesions, it alone can not be responsible for the observed increase in DKK-1 during PCa development. However, the data suggest that β-catenin could regulate DKK-1 in PCa cells as the latter decreased sharply as β-catenin shifted away from the nucleus in PCa metastases.

In the present invention it was shown that β-catenin is expressed at high levels in normal prostate, PIN, and primary lesions. β-catenin expression was also found to simultaneously decrease and redistribute from the nucleus to the cytoplasm in PCa metastases. Expression of β-catenin likely does not account for the increase in DKK-1 within primary lesions as β-catenin levels are similar in both normal and primary lesions. However, the reduction in DKK-1 in PCa metastases could be mediated in part by β-catenin as nuclear expression of β-catenin decreases in PCa metastases.

### Example 7

### DKK-1 is Expressed at Low Levels within Bone Metastases Compared to Soft Tissue Metastases

To evaluate the impact of organ site on DKK-1 expression, an autopsy array composed of soft tissue and skeletal metastases was evaluated. The autopsy TMA contained a total of 357 evaluable cores representing 10 types of metastatic lesion from 30 total PCa patients. The results showed that DKK-1 expression was higher in PCa primary lesions compared to the pooled PCa metastases that were a collection of lymph node and liver metastases. Evaluated individually, DKK-1 expression in PCa primary lesions was greater than each of PCa metastases of the lung, liver, bone, adrenal, and lymph node (primary PCa: mean, 105.9±15.6, median, 60; lung: mean, 99.5±17.9, median, 20; liver: mean, 83.9±15.4, median, 0; bone: mean, 66.7±13.4, median, 0; adrenal: mean 62.5±49.2, median, 0; lymph node, 53.8±12.7, median, 0). Interestingly, DKK-1 was found to be increased over PCa primary lesions in a number of soft tissue metastases including the bladder, dura, and seminal vesicles (bladder: mean 201.7±37.5, median, 180; dura: mean 154.3±36.2, median, 170; seminal vesicles: mean, 200±70.7, median, 250).

Transfection of DKK-1 into C4-2B PCa cells transformed these cells from an osteoblastic to a highly osteolytic tumor in vivo suggesting that PCa-induced osteoblastic response is mediated through Wnts. The data predict that DKK-1 expression should be decreased in clinical PCa bone metastases to allow for Wnt mediated bone formation. The clinical observations are in agreement with the experimental data provided herein which predict that DKK-1 expression decreases in PCa bone metastases to permit a Wnt-mediated osteoblastic reaction (Hall, 2005). Although it is not known whether high DKK-1 expression contributes to tumor growth in the bladder, dura, and seminal vesicles, the effect of low levels of DKK-1 expression within bone metastases would contribute to the formation of osteoblastic lesions through Wnts.

It has been demonstrated herein that DKK-1 is expressed early during PCa development. At present, it is unclear whether the increased DKK-1 expression results from or contributes to primary tumor development. Initial high DKK-1 expression within bone lesions could promote tumor establishment via a vicious cycle (Siclari, C M R 2006) by preventing Wnt-mediated suppression of osteoclastogenesis (Glass, 2005). Once the tumor has established in bone, subsequent reductions in DKK-1 expression observed in the autopsy array could permit Wnt-mediated bone formation which is characteristic of PCa osseous lesions. In this way, DKK-1 expression could explain the presence of both osteolytic and osteoblastic disease in PCa osseous lesions.

### Example 8

### High DKK-1 Levels are Associated with Shorter Overall Survival

Survival data from the patients represented in the autopsy TMA allowed evaluation of DKK-1 expression as a prognostic marker for PCa. A Kaplan-Meier blot comparing DKK-1 expression vs. patient survival from diagnosis to death was constructed. To compare DKK-1 expression, the tumor with the highest DKK-1 expression within a patient was selected to represent that patient. These DKK-1 max scores were then used to separate the patients between those with DKK-1 EI greater than 200 vs. those with DKK-1 EI less than 200. Of the 30 patients represented in the autopsy array, survival data was available for only 23 patients. The blot shows a near statistically significant trend (p<0.07) of high DKK-1 expression with shorter overall survival. Although there are examples of human tumors that over-express Wnt inhibitors to promote tumor growth, this is the first report to show that high levels of these inhibitors are associated with poor patient survival (Hall C M R, 2006). In this small study, DKK-1 was not found to be a prognostic marker, however, the elevated expression in PCa primary lesions and metastasis suggest that DKK-1 could be a suitable therapeutic target for PCa.

The observation that high DKK-1 expression in PCa metastases is associated with shorter patient survival seems inconsistent with the tumor promoting effects of Wnt signaling. Low levels of DKK-1 in soft tissue metastases of the liver, lung, lymph node and adrenal suggest that canonical Wnt signaling contributes more heavily to the development of lesions at these sites. Persistent high levels of DKK-1 expression in PCa soft tissue metastases suggest a functional role of DKK-1 in these lesions. In support of this hypothesis, it was recently reported that DKK-1 in PCa cells suppresses Wnt-mediated induction of osteoprotegerin and cyclin dependent kinase inhibitors p15 and p21 which have a dual role in suppressing tumor growth. These data are in agreement with published reports that show an elevated expression of Wnt inhibitors sFRP1, sFRP2, and DKK-1 in human tumors and that their expression can promote tumor growth in animal models (Wirths, 2003; Joesting, 2005; Oshima, 2005; Roth, 2000;). Taken together, the data support a mechanistic role of DKK-1 in PCa development and progression.

1. A method of reducing tumor burden from prostate cancer cells comprising the step of contacting the prostate cancer cells with a composition comprising an shRNA directed against DKK-1.

2. The method of claim 1, wherein the reduction in tumor burden is a reduction in metastasis of the prostate cancer cells.

3. The method of claim 1 wherein the composition is a vector encoding a shRNA directed against DKK-1, wherein the vector is taken up by prostate cancer cells in said patient and said shRNA is expressed in an amount sufficient to block the expression of DKK-1 in said prostate cancer cells.

4. The method of claim 1 further comprising the step of contacting said cancer cells with an anti-cancer agent.

5. The method of claim 1 wherein said shRNA is directed against a sequence selected from one or more of the sequences of SEQ ID NO:2, SEQ ID NO:3, SEQ ID NO:4, SEQ ID NO:5, SEQ ID NO: 10, SEQ ID NO:11, SEQ ID NO: 12, SEQ ID NO:13 and SEQ ID NO:14.

6. The method of claim 1 wherein said shRNA consists essentially of a sequence selected from the group consisting of SEQ ID NO:6, SEQ ID NO:7, SEQ ID NO:8, SEQ ID NO:9, SEQ ID NO: 15, SEQ ID NO:16, SEQ ID NO:17, SEQ ID NO:18 and SEQ ID NO:19.

7. A composition comprising a vector encoding an shRNA sequence directed against a DKK-1-encoding polynucleotide sequence selected from a group consisting of one or more of the sequences of SEQ ID NO:2, SEQ ID NO:3, SEQ ID NO:4, SEQ ID NO:5, SEQ ID NO:10, SEQ ID NO:11, SEQ ID NO:12, SEQ ID NO:13 and SEQ ID NO:14.

8. An shRNA selected from the group consisting of SEQ ID NO:6, SEQ ID NO:7, SEQ ID NO:8, SEQ ID NO:9, SEQ ID NO: 15, SEQ ID NO: 16, SEQ ID NO: 17, SEQ ID NO: 18 and SEQ ID NO: 19.

9. A mammalian host cell stably transfected with a vector that drives transcription of a polynucleotide encoding an shRNA sequence directed against a DKK-1-encoding polynucleotide sequence selected from a group consisting of one or more of the sequences of SEQ ID NO:2, SEQ ID NO:3, SEQ ID NO:4, SEQ ID NO:5, SEQ ID NO: 10, SEQ ID NO:11, SEQ ID NO:12, SEQ ID NO:13 and SEQ ID NO: 14.

10. A mammalian host cell stably transfected with a vector that drives transcription of a polynucleotide encoding an shRNA selected from the group consisting of SEQ ID NO:6, SEQ ID NO:7, SEQ ID NO:8, SEQ ID NO:9, SEQ ID NO: 15, SEQ ID NO:16, SEQ ID NO:17, SEQ ID NO:18 and SEQ ID NO:19.

11. A method for diagnosing prostate cancer comprising the step of detecting DKK-1 activity levels in a patient, wherein elevated levels compared to levels in a normal individual are suggestive of prostate cancer, and wherein said normal individual is known not to suffer from prostate cancer.

12-16. (canceled)

17. A method for determining susceptibility to prostate cancer in a patient, comprising the step of determining DKK-1 activity levels in said patient, wherein elevated DKK-1 activity levels in said patient compared to DKK-1 activity levels in a normal individual indicate susceptibility to prostate cancer, wherein said normal individual is known not to suffer from prostate cancer.

18. A method for determining the progression of prostate cancer in a patient, comprising the step of determining DKK-1 activity levels in said patient, wherein increasing levels over time are indicative of prostate cancer progression.

19. A method for monitoring the effectiveness of prostate cancer treatment comprising the step of measuring DKK-1 activity over time, wherein a decrease in a rate of DKK-1 activity increase is indicative of effective treatment.

20.-23. (canceled)

24. A method for determining susceptibility to bone cancer in a patient, comprising the step of determining DKK-1 activity in said patient, wherein elevated DKK-1 activity in said patient compared to DKK-1 activity in a normal individual indicates susceptibility to bone cancer, and wherein said normal individual is known not to suffer from bone cancer.

25. A method for determining susceptibility to bone cancer in a patient, comprising the step of determining DKK-1 activity in said patient, wherein elevated DKK-1 activity in said patient compared to prior levels in said patient indicates susceptibility to bone cancer.

26. (canceled)

27. (canceled)

28. (canceled)

29. (canceled)


SEQUENCE LISTING (84 sequences)

SEQ ID NO: 1 - DNA, 1815 bp, Homo sapiens
gcagagctctgtgctccctgcagtcaggactctgggaccgcagggggctcccggaccctgactctgcagccgaaccggcacggtttcgtggggacccaggcttgcaaagtgacggtcattttctctttctttctccctcttgagtccttctgagatgatggctctgggcgcagcgggagctacccgggtctttgtcgcgatggtagcggcggctctcggcggccaccctctgctgggagtgagcgccaccttgaactcggttctcaattccaacgctatcaagaacctgcccccaccgctgggcggcgctgcggggcacccaggctctgcagtcagcgccgcgccgggaatcctgtacccgggcgggaataagtaccagaccattgacaactaccagccgtacccgtgcgcagaggacgaggagtgcggcactgatgagtactgcgctagtcccacccgcggaggggacgcaggcgtgcaaatctgtctcgcctgcaggaagcgccgaaaacgctgcatgcgtcacgctatgtgctgccccgggaattactgcaaaaatggaatatgtgtgtcttctgatcaaaatcatttccgaggagaaattgaggaaaccatcactgaaagctttggtaatgatcatagcaccttggatgggtattccagaagaaccaccttgtcttcaaaaatgtatcacaccaaaggacaagaaggttctgtttgtctccggtcatcagactgtgcctcaggattgtgttgtgctagacacttctggtccaagatctgtaaacctgtcctgaaagaaggtcaagtgtgtaccaagcataggagaaaaggctctcatggactagaaatattccagcgttgttactgtggagaaggtctgtcttgccggatacagaaagatcaccatcaagccagtaattcttctaggcttcacacttgtcagagacactaaaccagctatccaaatgcagtgaactccttttatataatagatgctatgaaaaccttttatgaccttcatcaactcaatcctaaggatatacaagttctgtggtttcagttaagcattccaataacaccttccaaaaacctggagtgtaagagctttgtttctttatggaactcccctgtgattgcagtaaattactgtattgtaaattctcagtgtggcacttacctgtaaatgcaatgaaacttttaattatttttctaaaggtgctgcactgcctatttttcctcttgttatgtaaatttttgtacacattgattgttatcttgactgacaaatattctatattgaactgaagtaaatcatttcagcttatagttcttaaaagcataaccctttaccccatttaattctagagtctagaacgcaaggatctcttggaatgacaaatgataggtacctaaaatgtaacatgaaaatactagcttattttctgaaatgtactatcttaatgcttaaattatatttccctttaggctgtgatagtttttgaaataaaatttaacatttaatatcatgaaatgttataagtagacatacattttgggattgtgatcttagaggtttgtgtgtgtgtacgtatgtgtgtgttctacaagaacggaagtgtgatatgtttaaagatgatcagagaaaagacagtgtctaaatataagacaatattgatcagctctagaataactttaaagaaagacgtgttctgcattgataaactcaaatgatcatggcagaatgagagtgaatcttacattactactttcaaaaatagtttccaataaattaataatacctaaaaaaaaaaa

SEQ ID NO: 2 - DNA, 21 bp, Artificial sequence
  Synthetic polynucleotide
ggaataagtaccagaccattg

SEQ ID NO: 3 - DNA, 21 bp, Artificial sequence
  Synthetic polynucleotide
gctctcatggactagaaatat

SEQ ID NO: 4 - DNA, 21 bp, Artificial sequence
  Synthetic polynucleotide
ggagtgtaagagctttgtttc

SEQ ID NO: 5 - DNA, 21 bp, Artificial sequence
  Synthetic polynucleotide
ggatctcttggaatgacaaat

SEQ ID NO: 6 - DNA, 21 bp, Artificial sequence
  Synthetic polynucleotide
ccttattcatggtctggtaag

SEQ ID NO: 7 - DNA, 21 bp, Artificial sequence
  Synthetic polynucleotide
cgagagtacctgatctttata

SEQ ID NO: 8 - DNA, 21 bp, Artificial sequence
  Synthetic polynucleotide
cctcacattctcgaaacaaag

SEQ ID NO: 9 - DNA, 21 bp, Artificial sequence
  Synthetic polynucleotide
cctagagaaccttactgttta

SEQ ID NO: 10 - DNA, 21 bp, Artificial sequence
  Synthetic polynucleotide
ggttctcaattccaacgctat

SEQ ID NO: 11 - DNA, 21 bp, Artificial sequence
  Synthetic polynucleotide
ggtaatgatcatagcaccttg

SEQ ID NO: 12 - DNA, 21 bp, Artificial sequence
  Synthetic polynucleotide
gctagacacttctggtccaag

SEQ ID NO: 13 - DNA, 21 bp, Artificial sequence
  Synthetic polynucleotide
gccggatacagaaagatcacc

SEQ ID NO: 14 - DNA, 21 bp, Artificial sequence
  Synthetic polynucleotide
gcacttacctgtaaatgcaat

SEQ ID NO: 15 - DNA, 21 bp, Artificial sequence
  Synthetic polynucleotide
ccaagagttaaggttgcgata

SEQ ID NO: 16 - DNA, 21 bp, Artificial sequence
  Synthetic polynucleotide
ccattactagtatcgtggaac

SEQ ID NO: 17 - DNA, 21 bp, Artificial sequence
  Synthetic polynucleotide
cgatctgtgaagaccaggttc

SEQ ID NO: 18 - DNA, 21 bp, Artificial sequence
  Synthetic polynucleotide
cggcctatgtctttctagtgg

SEQ ID NO: 19 - DNA, 21 bp, Artificial sequence
  Synthetic polynucleotide
cgtgaatggacatttacgtta

SEQ ID NO: 20 - DNA, 20 bp, Artificial sequence
  Synthetic primer
ggcaacacagctcacaagaa

SEQ ID NO: 21 - DNA, 20 bp, Artificial sequence
  Synthetic primer
ctgggtttgcatgcctttat

SEQ ID NO: 22 - DNA, 20 bp, Artificial sequence
  Synthetic primer
gaccgggaataaccttccat

SEQ ID NO: 23 - DNA, 20 bp, Artificial sequence
  Synthetic primer
caccaggtccagtcaaggat

SEQ ID NO: 24 - DNA, 20 bp, Artificial sequence
  Synthetic primer
atgaaattcaccccctttcc

SEQ ID NO: 25 - DNA, 20 bp, Artificial sequence
  Synthetic primer
ccctaggctgtgctcacttc

SEQ ID NO: 26 - DNA, 20 bp, Artificial sequence
  Synthetic primer
acccaatccctctccactct

SEQ ID NO: 27 - DNA, 20 bp, Artificial sequence
  Synthetic primer
gattcaaggaaaagccacca

SEQ ID NO: 28 - DNA, 20 bp, Artificial sequence
  Synthetic primer
gtggatgcaaaggaaaggaa

SEQ ID NO: 29 - DNA, 20 bp, Artificial sequence
  Synthetic primer
agccagcatgtcctgagagt

SEQ ID NO: 30 - DNA, 20 bp, Artificial sequence
  Synthetic primer
gtgtcctggctggttcctta

SEQ ID NO: 31 - DNA, 20 bp, Artificial sequence
  Synthetic primer
agctggtgcaaaggaaagaa

SEQ ID NO: 32 - DNA, 20 bp, Artificial sequence
  Synthetic primer
tgtgaggtgaagacctgctg

SEQ ID NO: 33 - DNA, 20 bp, Artificial sequence
  Synthetic primer
aaagttgggggagttctcgt

SEQ ID NO: 34 - DNA, 20 bp, Artificial sequence
  Synthetic primer
ggacaaagctaccagggagt

SEQ ID NO: 35 - DNA, 20 bp, Artificial sequence
  Synthetic primer
actcgatgtcctcgctacag

SEQ ID NO: 36 - DNA, 20 bp, Artificial sequence
  Synthetic primer
ctcatgaacctccacaacaa

SEQ ID NO: 37 - DNA, 20 bp, Artificial sequence
  Synthetic primer
gcaccatcaaacttctcctt

SEQ ID NO: 38 - DNA, 20 bp, Artificial sequence
  Synthetic primer
cttggtggtcgctaggtatg

SEQ ID NO: 39 - DNA, 20 bp, Artificial sequence
  Synthetic primer
tcggaattgatactggcatt

SEQ ID NO: 40 - DNA, 20 bp, Artificial sequence
  Synthetic primer
gggctttttctctccctctg

SEQ ID NO: 41 - DNA, 20 bp, Artificial sequence
  Synthetic primer
cgaggttgaagctgagttcc

SEQ ID NO: 42 - DNA, 20 bp, Artificial sequence
  Synthetic primer
gtcacgcaggcctgttctat

SEQ ID NO: 43 - DNA, 20 bp, Artificial sequence
  Synthetic primer
cgtccataaagagcctcgac

SEQ ID NO: 44 - DNA, 20 bp, Artificial sequence
  Synthetic primer
tctcatgaacttgcacaaca

SEQ ID NO: 45 - DNA, 20 bp, Artificial sequence
  Synthetic primer
acttgtccttgagcacgtag

SEQ ID NO: 46 - DNA, 20 bp, Artificial sequence
  Synthetic primer
gcctgcaggtcctagaagtg

SEQ ID NO: 47 - DNA, 20 bp, Artificial sequence
  Synthetic primer
ctcccaaagtgctgggatta

SEQ ID NO: 48 - DNA, 20 bp, Artificial sequence
  Synthetic primer
gaactgccctgaaaatgctc

SEQ ID NO: 49 - DNA, 20 bp, Artificial sequence
  Synthetic primer
atcctttccccaaattccac

SEQ ID NO: 50 - DNA, 20 bp, Artificial sequence
  Synthetic primer
ccatgaacctgcacaacaac

SEQ ID NO: 51 - DNA, 20 bp, Artificial sequnce
  Synthetic primer
tgagtgctgcgtggtacttc

SEQ ID NO: 52 - DNA, 20 bp, Artificial sequence
  Synthetic primer
gcaagcatctgaagcacaag

SEQ ID NO: 53 - DNA, 20 bp, Artificial sequence
  Synthetic primer
tgctctcgcagttcttctca

SEQ ID NO: 54 - DNA, 20 bp, Artificial sequence
  Synthetic primer
gaggactcacccagcttctg

SEQ ID NO: 55 - DNA, 20 bp, Artificial sequence
  Synthetic primer
taggcctagtgcttgcaggt

SEQ ID NO: 56 - DNA, 20 bp, Artificial sequence
  Synthetic primer
ggttgctccacaccctaaaa

SEQ ID NO: 57 - DNA, 20 bp, Artificial sequence
  Synthetic primer
atgatgaagggaatggtgga

SEQ ID NO: 58 - DNA, 20 bp, Artificial sequence
  Synthetic primer
agttctctcgggatttcttg

SEQ ID NO: 59 - DNA, 20 bp, Artificial sequence
  Synthetic primer
cgcttcaggttttcagttac

SEQ ID NO: 60 - DNA, 20 bp, Artificial sequence
  Synthetic primer
cccaagccaataaactgatg

SEQ ID NO: 61 - DNA, 20 bp, Artificial sequence
  Synthetic primer
aggtatcgggtcttgaggtc

SEQ ID NO: 62 - DNA, 20 bp, Artificial sequence
  Synthetic primer
gctcctgtgctgtgaaaaca

SEQ ID NO: 63 - DNA, 20 bp, Artificial sequence
  Synthetic primer
tgcattctctgccttgtgtc

SEQ ID NO: 64 - DNA, 20 bp, Artificial sequence
  Synthetic primer
gtcctcaaggtgccatccta

SEQ ID NO: 65 - DNA, 20 bp, Artificial sequence
  Synthetic primer
cagcccgacagaaaaatgat

SEQ ID NO: 66 - DNA, 20 bp, Artificial sequence
  Synthetic primer
ctctctttggcccttgactg

SEQ ID NO: 67 - DNA, 20 bp, Artificial sequence
  Synthetic primer
acaaagaaaaggccggaaat

SEQ ID NO: 68 - DNA, 20 bp, Artificial sequence
  Synthetic primer
ttctggataggcctgtggtc

SEQ ID NO: 69 - DNA, 20 bp, Artificial sequence
  Synthetic primer
cgtagtggatgtggttgtgc

SEQ ID NO: 70 - DNA, 20 bp, Artificial sequence
  Synthetic primer
ttgttggcatctctgctgtc

SEQ ID NO: 71 - DNA, 20 bp, Artificial sequence
  Synthetic primer
ccatggatttggaaatgacc

SEQ ID NO: 72 - DNA, 20 bp, Artificial sequence
  Synthetic primer
gccatcgactatgacccact

SEQ ID NO: 73 - DNA, 20 bp, Artificial sequence
  Synthetic primer
cagaacagtgtccggctgta

SEQ ID NO: 74 - DNA, 20 bp, Artificial sequence
  Synthetic primer
cccatgcacctggttctact

SEQ ID NO: 75 - DNA, 20 bp, Artificial sequence
  Synthetic primer
ctggaactgggactctgagc

SEQ ID NO: 76 - DNA, 20 bp, Artificial sequence
  Synthetic primer
tagcaccttggatgggtatt

SEQ ID NO: 77 - DNA, 20 bp, Artificial sequence
  Synthetic primer
atcctgaggcacagtctgat

SEQ ID NO: 78 - DNA, 20 bp, Artificial sequence
  Synthetic primer
gatgtgctccagtgactttg

SEQ ID NO: 79 - DNA, 20 bp, Artificial sequence
  Synthetic primer
gcttgagcagcttcttcttt

SEQ ID NO: 80 - DNA, 20 bp, Artificial sequence
  Synthetic primer
ccaaggtcatccatgacaac

SEQ ID NO: 81 - DNA, 20 bp, Artificial sequence
  Synthetic primer
agaggcagggatgatgttct

SEQ ID NO: 82 - DNA, 20 bp, Artificial sequence
  Synthetic primer
ggacttcgagcaagagatgg

SEQ ID NO: 83 - DNA, 20 bp, Artificial sequence
  Synthetic primer
agcactgtgttggcgtacag

SEQ ID NO: 84 - DNA, 50 bp, Homo sapiens
caatggtctggtacttattcccgaaggaataagtaccagaccattgcacc